(19) United States
(12) Patent Application Publication
(10) Pub. No.: US 2018/0153849 A1
(43) Pub. Date: Jun. 7, 2018

(54) MULTI-DRUG COMBINATIONS THAT ACT AS POTENT RADIOSENSITIZERS

(71) Applicant: Wisconsin Alumni Research Foundation, Madison, WI (US)

(72) Inventors: Glen S. KWON, Madison, WI (US); Keishiro TOMODA, Madison, WI (US); Kevin KOZAK, Madison, WI (US)

(21) Appl. No.: 15/735,493

(22) PCT Filed: Jun. 10, 2016
(86) PCT No.: PCT/US2016/037096

§ 371 Date: Dec. 11, 2017

Related U.S. Application Data
(60) Provisional application No. 62/175,166, filed on Jun. 12, 2015.

(51) Int. Cl. A61K 31/337; A61K 31/436; A61K 9/107; A61P 35/00

(52) U.S. Cl. CPC A61K 31/337; A61K 31/436; A61K 9/19; A61P 35/00; A61K 2300/00; A61K 9/1075

(57) ABSTRACT

Provided is a method of treatment including administering to lung, brain or esophageal cancer cells an effective amount of radiation in conjunction with a micelle composition that includes an effective amount of paclitaxel, and one or both of 17-AAG, and rapamycin, wherein the micelle includes poly(ethylene glycol)-block-poly(lactic acid) (PEG-b-PLA).

### CROSS-REFERENCE TO RELATED APPLICATIONS

This application claims priority to U.S. Provisional Patent Application No. 61/175,166, filed on Jun. 12, 2015, the entire contents of which are incorporated herein by reference.

### STATEMENT OF GOVERNMENT SUPPORT

This invention was made with government support under AI101157 awarded by the National Institutes of Health. The United States government has certain rights in the invention.

### FIELD

The present technology relates generally to combination drug and radiation therapy treatment of lung cancer, and is useful for inhibiting lung cancer cell proliferation and tumor proliferation.

### SUMMARY

The present technology provides methods for the treatment of lung cancer and other cancers employing a combination of multi-drug chemotherapy and radiation therapy. Surprisingly, it has been found that micelles including multi-drug combinations of paclitaxel, 17-allylamino-17-dimethoxy-geldanamycin (17-AAG), and/or rapamycin are potent radiosensitizers of lung cancer cells in vivo. The micelles include poly(ethylene glycol)-block-poly(lactic acid) (PEG-b-PLA) and are highly effective at solubilizing such hydrophobic anti-cancer drugs. Various lung cancers may be treated by the present methods, including non-small cell lung cancers such as squamous cell carcinoma, adenocarcinoma, and large cell carcinoma. In addition, brain cancer and esophageal cancer may be treated using the present methods.

In particular, the present methods include administering to lung, brain or esophageal cancer cells an effective amount of radiation in conjunction with a micelle composition that includes an effective amount of a multi-drug combination of anticancer drugs. The micelles comprise poly(ethylene glycol)-block-poly(lactic acid) and may contain two or three of paclitaxel, 17-AAG, and rapamycin. These micelles provide improved radiosensitization of the cancer cells compared to micelles prepared from the same polymer and paclitaxel alone. In certain embodiments, the micelle composition may contain a ratio by mass of paclitaxel, 17-AAG, and rapamycin of about 2:2:1 to about 5:1:1. For example, the micelle composition may have a 2:2:1 ratio by mass of about 2-80 mg/kg of paclitaxel, 2-80 mg/kg of 17-AAG, and 1-40 mg/kg of rapamycin. In some embodiments, the micelle composition includes a drug load of paclitaxel, and one or both 17-AAG and rapamycin of about 5 wt. % to about 40 wt. %. The micelles of the present methods include PEG-b-PLA wherein the poly(ethylene glycol) block may have a molecular weight of about 1,500 g/mol to about 30,000 g/mol, and the poly(lactic acid) block may have a molecular weight of about 1,500 to about 30,000 g/mol.

In the present methods, the radiation and micelle composition may be administered to the lung (or brain or esophageal) cancer cells sequentially, simultaneously, or separately. The radiation therapy may, e.g., be administered at dosages about 1 to about 8 Gy/day and/or over a period of about 1 to about 5 days. The present methods also include administration to the cancer cells in vitro or in vivo, where in vivo administration can be to lung (or brain or esophageal) cancer cells in a subject, for example, a human subject. The micelle composition, including an aqueous carrier such as saline or glucose solution, may intravenously or intraperitoneally administered to the subject with lung, brain or esophageal cancer. Thus, in some aspects, the present methods include administering to a subject having lung, brain or esophageal cancer an effective amount of radiation in conjunction with a micelle composition that includes paclitaxel, 17-AAG, and/or rapamycin.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIG. 1 shows in vitro clonogenic assay results, illustrating survival rates for A549 non-small cell lung cancer adenocarcinoma (A549 cells) treated with one, two, or three drug-loaded micelles, containing the drugs paclitaxel (PTX), 17-AAG, and/or rapamycin (RAP), with radiation treatment (XRT).

FIG. 2 shows in vitro clonogenic assay results, illustrating the surviving fraction of A549 cells treated with one, two or three drug-loaded micelles, containing the drugs paclitaxel (PTX), 17-AAG, and/or rapamycin (RAP), as a function of radiation (XRT) dosage (0-8 Gy).

FIG. 3 shows in vivo anticancer efficacy for A549 cell inoculated mice treated with paclitaxel-loaded micelle composition with and without irradiation (XRT), illustrating the rate in relative tumor volume growth as a function of time (days).

FIG. 4 shows in vivo drug toxicity effects for A549 cell inoculated mice treated with (60 mg/kg) paclitaxel-loaded micelle composition or vehicle (only PEG-b-PLA micelle composition at 105 mg/kg) with and without irradiation (XRT), illustrating the growth rate in relative body weight as a function of time (days).

FIG. 5 shows in vivo anticancer efficacy for A549 cell inoculated mice treated with high dosage (60/60/30 mg/kg) paclitaxel/17-AAG/rapamycin loaded micelle composition (Triolimus) or vehicle (only PEG-b-PLA micelle composition at 105 mg/kg) with and without irradiation (XRT), illustrating the rate in relative tumor volume growth as a function of time (days).

FIG. 6 shows in vivo drug toxicity effects for A549 cell inoculated mice treated with high dosage (60/60/30 mg/kg) paclitaxel/17-AAG/rapamycin loaded micelle composition (Triolimus) or vehicle (only PEG-b-PLA micelle composition at 105 mg/kg) with and without irradiation (XRT), illustrating the growth rate in mice relative body weight as a function of time (days).

FIG. 7 shows in vivo anticancer efficacy for A549 cell inoculated mice treated with low dosage (15/15/7.5 mg/kg) paclitaxel/17-AAG/rapamycin loaded micelle composition (Triolimus) or vehicle (only PEG-b-PLA micelle composition at 105 mg/kg) with and without irradiation (XRT), illustrating the rate in relative tumor volume growth as a function of time (days).

FIG. 8 shows in vivo drug toxicity effects for A549 cell inoculated mice treated with low dosage (15/15/7.5 mg/kg) paclitaxel/17-AAG/rapamycin loaded micelle composition (Triolimus) or vehicle (only PEG-b-PLA micelle at 105 mg/kg) with and without irradiation (XRT), illustrating the growth rate in mice relative body weight as a function of time (days).

FIG. 9 shows in vivo anticancer efficacy for A549 cell inoculated mice treated with (60/60 mg/kg) paclitaxel (PTX)/17-AAG-loaded micelle composition or vehicle (only PEG-b-PLA micelle composition at 105 mg/kg) with and without irradiation (XRT), illustrating the rate in relative tumor volume growth as a function of time (days).

FIG. 10 shows in vivo drug toxicity effects for A549 cell inoculated mice treated with (60/60 mg/kg) paclitaxel (PTX)/17-AAG-loaded micelle composition or vehicle (only PEG-b-PLA micelle composition at 105 mg/kg) with and without irradiation (XRT), illustrating the growth rate in mice relative body weight as a function of time (days).

FIG. 11 shows in vivo anticancer efficacy for A549 cell inoculated mice treated with (60/30 mg/kg) paclitaxel (PTX)/rapamycin (RAP)-loaded micelle composition or vehicle (only PEG-b-PLA micelle composition at 105 mg/kg) with and without irradiation (XRT), illustrating the rate in relative tumor volume growth as a function of time (days).

FIG. 12 shows in vivo drug toxicity effects for A549 cell inoculated mice treated with (60/30 mg/kg) paclitaxel (PTX)/rapamycin (RAP)-loaded micelle composition or vehicle (only PEG-b-PLA micelle composition at 105 mg/kg) with and without irradiation (XRT), illustrating the growth rate in mice relative body weight as a function of time (days).

FIG. 13 shows in vivo anticancer efficacy for A549 cell inoculated mice treated (60/30 mg/kg) 17-AAG/rapamycin (RAP)-loaded micelle composition or vehicle (only PEG-b-PLA micelle composition at 105 mg/kg) with and without irradiation (XRT), illustrating the rate in relative tumor volume growth as a function of time (days).

FIG. 14 shows in vivo drug toxicity effects for A549 cell inoculated mice treated with (60/30 mg/kg) 17-AAG/rapamycin (RAP)-loaded micelle composition or vehicle (only PEG-b-PLA micelle composition at 105 mg/kg) with and without irradiation (XRT), illustrating the growth rate in mice relative body weight as a function of time (days).

FIG. 15 shows in vitro clonogenic assay results, illustrating survival rates for A549 cells treated with 1 nM, 2 nM, and 3 nM Triolimus drug-loaded micelles.

FIG. 16 shows in vitro clonogenic assay results, illustrating survival rates for A549 cells treated with 1 nM and 2 nM Triolimus drug-loaded micelles or no treatment, as a function of radiation (XRT) dosage (0-8 Gy).

FIG. 17 shows tumor necrosis for A549 cell inoculated mice treated with (60 mg/kg) PTX, (60/60 mg/kg) P/17, (60/30 mg/kg) P/R, (60/30 mg/kg) 17/R loaded micelle compositions or vehicle (only PEG-b-PLA micelle at 105 mg/kg).

FIG. 18 shows tumor necrosis for A549 cell inoculated mice treated with (PTX/17-AAG/RAP: 2.4/2.4/1.2 mg/kg) Low TRIO, (PTX/17-AAG/RAP: 12/12/6 mg/kg) Int. TRIO, and (PTX/17-AAG/RAP: 60/60/30 mg/kg) High TRIO loaded micelle compositions or vehicle (only PEG-b-PLA micelle at 105 mg/kg).

FIG. 19 shows tumor necrosis for A549 cell inoculated mice treated with (PTX/17-AAG/RAP: 60/60/30 mg/kg) TRIO loaded micelle composition day 1 and euthanized day 1 (TRIO day 1); TRIO loaded micelle composition day 1, fractionated radiation for 3 Gy for five days (XRT), and euthanized day 6 (TRIO+XRT); TRIO loaded micelle composition day 1, no radiation, and euthanized day 6 (TRIO day 6); vehicle (only PEG-b-PLA micelle at 105 mg/kg); or vehicle+XRT.

FIG. 20 shows Ki-67 proliferative index for A549 cell inoculated mice treated with (60 mg/kg) PTX, (60/60 mg/kg) P/17, (60/30 mg/kg) P/R, (60/30 mg/kg) 17/R loaded micelle compositions or vehicle (only PEG-b-PLA micelle at 105 mg/kg).

FIG. 21 shows Ki-67 proliferative index for A549 cell inoculated mice treated with (PTX/17-AAG/RAP: 2.4/2.4/1.2 mg/kg) Low TRIO, (PTX/17-AAG/RAP: 12/12/6 mg/kg) Int. TRIO, and (PTX/17-AAG/RAP: 60/60/30 mg/kg) High TRIO loaded micelle compositions or vehicle (only PEG-b-PLA micelle at 105 mg/kg).

FIG. 22 shows Ki-67 proliferative index for A549 cell inoculated mice treated with (PTX/17-AAG/RAP: 60/60/30 mg/kg) TRIO loaded micelle composition day 1 and euthanized day 1 (TRIO day 1); TRIO loaded micelle composition day 1, fractionated radiation for 3 Gy for five days (XRT), and euthanized day 6 (TRIO+XRT); TRIO loaded micelle composition day 1, no radiation, and euthanized day 6 (TRIO day 6); vehicle (only PEG-b-PLA micelle at 105 mg/kg); or vehicle+XRT.

FIG. 23 shows levels of cleaved caspase 3 as a measure of apoptosis for A549 cell inoculated mice treated with (60 mg/kg) PTX, (60/60 mg/kg) P/17, (60/30 mg/kg) P/R, (60/30 mg/kg) 17/R loaded micelle compositions or vehicle (only PEG-b-PLA micelle at 105 mg/kg).

FIG. 24 shows levels of cleaved caspase 3 as a measure of apoptosis for A549 cell inoculated mice treated with (PTX/17-AAG/RAP: 2.4/2.4/1.2 mg/kg) Low TRIO, (PTX/17-AAG/RAP: 12/12/6 mg/kg) Int. TRIO, and (PTX/17-AAG/RAP: 60/60/30 mg/kg) High TRIO loaded micelle compositions or vehicle (only PEG-b-PLA micelle at 105 mg/kg).

FIG. 25 shows levels of cleaved caspase 3 as a measure of apoptosis for A549 cell inoculated mice treated with (PTX/17-AAG/RAP: 60/60/30 mg/kg) TRIO loaded micelle composition day 1 and euthanized day 1 (TRIO day 1); TRIO loaded micelle composition day 1, fractionated radiation for 3 Gy for five days (XRT), and euthanized day 6 (TRIO+XRT); TRIO loaded micelle composition day 1, no radiation, and euthanized day 6 (TRIO day 6); vehicle (only PEG-b-PLA micelle at 105 mg/kg); or vehicle+XRT.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology provides new methods of treating lung, brain or esophageal cancer that take advantage of the surprising effect of combining radiation therapy and multi-drug combinations of anti-cancer agents delivered by certain types of micelles. Poly(ethylene glycol)-block-poly(lactic acid) (PEG-b-PLA) forms a micellar multi-drug delivery system, that is advantageous for, but not limited to, poorly water soluble drugs. For example, PEG-b-PLA can safely deliver two- and three drug combinations of paclitaxel, 17-AAG, and rapamycin in lung cancer treatments (e.g., Triolimus or TRIO, which contains all three drugs). It has now been discovered that combinations of paclitaxel, 17-AAG, and/or rapamycin in conjunction with radiation therapy provide larger anti-cancer effects than combined radiation therapy and micelle-delivery of paclitaxel alone. Certain combinations such as paclitaxel, 17-AAG and rapamycin in conjunction with radiation therapy may not only slow tumor growth but stop or reverse it all together.

Thus, the present technology provides methods of treating lung cancer or inhibiting lung cancer cell proliferation that include administering an effective amount of radiation therapy in conjunction with a micelle composition which includes an effective amount of paclitaxel, 17-AAG, and rapamycin, wherein the micelle composition comprises poly(ethylene glycol)-block-poly(lactic acid). In some embodiments, the present methods include treating non-small cell lung cancer cells, including but not limited to squamous cell, adenocarcinoma, and large cell lung cancers. In other embodiments, the present methods include treating small cell lung cancer.

One-, two-, and three-drug formulations of rapamycin, paclitaxel, and/or 17-AAG can be prepared using amphiphilic diblock polymers such as PEG-b-PLA as set forth, e.g., in U.S. Pat. No. 8,858,965 (incorporated by reference in its entirety herein). While not wishing to be bound by theory, it is believed that the hydrophobic poly(lactic acid) block of the polymers orient toward the interior of each micelle, and the hydrophilic poly(ethylene glycol) block of the polymers orient toward the exterior of each micelle. The drugs described herein are non-covalently associated with the micelles such that the drugs are solubilized by the micelles, thereby forming drug delivery systems. For example, the drugs described herein may be encapsulated by the micelles, and/or present in and/or on the micelle membrane.

In the present methods, each of the polymer blocks of the PEG-b-PLA which forms the micelles, may encompass a range of molecular weights so long as the resulting polymer is capable of forming micelles in aqueous solution. For example, the molecular weight of the poly(ethylene glycol) block can be about 1,500 to about 30,000 g/mol, and the molecular weight of the poly(lactic acid) block can be about 1,500 to about 30,000 g/mol. Suitable molecular weights for the poly(ethylene glycol) block include about 1,500, about 2,000, about 3,000, about 4,000, about 5,000, about 6,000, about 8,000, about 10,000, about 12,000, about 14,000, about 20,000, about 25,000, or about 30,000 g/mol, or a range between and including any two of the foregoing values. Suitable molecular weights for the poly(lactic acid) block include about 1,500, about 2,000, about 3,000, about 4,000, about 5,000, about 6,000, about 7,000, about 10,000, about 12,000, about 14,000, about 20,000, about 25,000, or about 30,000 g/mol, or a range between and including any two of the foregoing values. In some embodiments of the present technology, the molecular weight of the poly(ethylene glycol) block can be about 4,000 to about 10,000 g/mol, and the molecular weight of the poly(lactic acid) block can be about 2,000 to about 5,000 g/mol. In certain embodiments, the molecular weight of the poly(ethylene glycol) block can be about 10,000 to about 14,000 g/mol, and the molecular weight of the poly(lactic acid) block can be about 5,000 to about 7,000 g/mol. Examples of suitable combinations of molecular weights for PEG-b-PLA include, but are not limited to, about 2,000-2,000 (2K-2K), about 3K-5K, about 5K-3K, about 5K-6K, about 6K-5K, about 6K-6K, about 8K-4K, about 4K-7K, about 7K-3K, about 3K-7K, about 12K-6K, and/or about 6K-7K g/mol.

The combined drug loading of micelles used in the present methods can be about 5 wt. % to about 50 wt. % with respect to the weight of the micelles (i.e., wt. % is equal to (combined weight of the drugs)/(wt of the polymers)×100). Examples of such combined drug loadings include about 5 wt %, about 10 wt %, about 15 wt %, about 20 wt %, about 25 wt %, about 30 wt %, about 35 wt %, about 40 wt %, about 45 wt %, about 50 wt %, or a range between and including any of the foregoing individual values. Thus, in some embodiments, the combined drug loading is about 5 wt. % to about 40 wt. %, about 5 wt. % to about 30 wt. %, about 10 wt % to about 40 wt %, about 10 wt % to about 30 wt %, about 10 wt. % to about 20 wt. %, or about 20 wt % to about 40 wt %.

Subject to the above combined drug loading limits, the drug loading in the micelles for each individual drug, can be about 1 wt % to about 25 wt %, with respect to the weight of the micelles. The drug loading of each drug can also be about 4 wt. % to about 24 wt %, or about 5 wt % to about 20 wt %, or about 6 wt % to about 15 wt %, with respect to the weight of the micelles. Examples of individual drug loadings include about 1 wt %, about 2 wt %, about 3 wt %, about 4 wt %, about 5 wt %, about 6 wt %, about 8 wt %, about 10 wt %, about 15wt %, about 20 wt %, about 21 wt %, about 22 wt %, about 23 wt %, about 24 wt % or about 25 wt %, or a range between and including any two of the foregoing values.

The weight ratio of drugs in the micelles can vary and may be routinely adjusted by those of skill in the art taking into account the present disclosure and the particular cancer, nature of the drugs used, types and sizes of micelles, the weight, age and condition of the patient, and other relevant parameters known in the art. For example, the weight ratio of two drugs in micelles (e.g., paclitaxel/rapamycin, paclitaxel/17-AAG and rapamycin/17-AAG) can be about 20:1 to about 1:20. Suitable two drug ratios include about 20:1, about 15:1 about 10:1, about 5:1, about 3:1, about 2:1, about 1:1, about 1:2, about 1:3, about 1:5, about 1:10, about 1:15, about 1:20, or a range between and including any two of the foregoing ratios. Similarly, the weight ratio of three drugs in micelles (e.g., paclitaxel, 17-AAG, and rapamycin) in the present methods, can vary such that any of the drugs may have a weight ratio up to 10 times any of the other components. Examples of suitable three-drug ratios by mass of paclitaxel, 17-AAG, and rapamycin in the PEG-b-PLA micelles include about 1:1:10, about 1:1:5, about 1:2:2, about 2:1:2, about 2:2:1, about 3:2:1, about 3:1:2, about 2:1:3, about 1:5:1, about 1:10:1, about 5:1:1, about 1:1:1, about 1:1:10 or a range between or including any of the foregoing ratios. In some embodiments, the weight ratio of paclitaxel, 17-AAG, and rapamycin is about 2:2:1 to about 5:1:1.

The amount of each of drug (e.g., paclitaxel, 17-AAG, and rapamycin) being administered via micelle compositions to a subject with lung, brain or esophageal cancer can be about 1 mg/kg (drug wt/subject wt) to about 100 mg/kg, or about 2 mg/kg to about 80 mg/kg. The drugs may be delivered in amounts according to the micelle drug loading ratios above. For example, at a drug loading ratio of 2:2:1, micelle compositions may be prepared to deliver about 2 to about 80 mg/kg of paclitaxel, about 2 to about 80 mg/kg of 17-AAG, and about 1 to about 40 mg/kg of rapamycin in the prescribed ratio. In some embodiments, about 40 to about 80 mg/kg of paclitaxel, about 40 to about 80 mg/kg of 17-AAG, and about 20 to about 40 mg/kg of rapamycin may be administered to the subject. Examples of suitable amounts of drugs to be delivered having a 2:2:1 ratio by mass include, but are not limited to, about 2/2/1 mg/kg of paclitaxel/17-AAG/rapamycin, about 15/15/7.5 mg/kg of paclitaxel/17-AAG/rapamycin, about 30/30/15 mg/kg of paclitaxel/17-AAG/rapamycin, about 50/50/25 gm/kg of paclitaxel/17-AAG/rapamycin, about 60/60/30 mg/kg of paclitaxel/17-AAG/rapamycin, or about 70/70/35 mg/kg of paclitaxel/17-AAG/rapamycin.

The drugs can be incorporated together into individual PEG-PLA micelles, thereby forming multiple drug micelles (MDM). Alternatively, the drugs can be incorporated individually into PEG-PLA micelles, thereby forming single drug micelles (SDM). Single drug micelles that contain different drugs can then be combined to provide a single drug micelle drug combination (SDMDC) composition, and the micelles can be combined in a single aqueous vehicle to provide a therapeutic drug delivery formulation.

The micelle composition can include an aqueous vehicle, wherein the concentration of the drugs is about 0.4 mg/mL to about 25 mg/mL, about 0.6 mg/mL to about 20 mg/mL, or about 1 mg/mL to about 15 mg/mL, with respect to the volume of the aqueous vehicle. The encapsulated drugs can have an aqueous solubility of about 1 mg/mL to about 10 mg/mL when contacted with an aqueous environment.

The micelle composition can be substantially free of pharmaceutically acceptable organic solvents, including but not limited to, ethanol, dimethyl sulfoxide, castor oil, and castor oil derivatives. For example, the micelle composition can comprise less than about 2 wt. %, less than about 1 wt. %, less than about 0.5 wt. %, or less than about 0.1 wt. %, of organic solvents such as, but not limited to, ethanol, dimethyl sulfoxide, castor oil, and castor oil derivatives, individually or in combination.

In the present methods, the radiation and the micelle composition may be administered sequentially, simultaneously, or separately to the lung, brain or esophageal cancer cells. For example, radiation therapy may be administered to the lung, brain or esophageal cancer cells after pretreatment of the cells with the micelle composition including paclitaxel, 17-AAG, and/or rapamycin. The radiation therapy may also be administered to lung, brain or esophageal cancer cells before administration of paclitaxel, 17-AAG, and/or rapamycin-loaded micelles. Additionally, the present methods can also include administering radiation therapy simultaneously, i.e., concurrently or overlapping, with administration of paclitaxel, 17-AAG, and/or rapamycin-loaded micelle. The methods of the present technology can also include administering radiation therapy to lung, brain or esophageal cancer cells separately from administration of the micelle composition paclitaxel, 17-AAG, and/or rapamycin-loaded micelle, such as days or weeks before or after administration of the micelle composition.

The present methods employ radiation therapy in conjunction with delivery of multi-drug combinations using micelles. In some aspects of the present methods, an effective amount of radiation therapy can be administered to lung, brain or esophageal cancer cells in dosages in the range of about 1 unit (Grays) per day (Gy/day) to about 8 Gy/day. In certain embodiments higher dosages of 9-10 Gy/day may be employed. Dosages of radiation therapy can also vary from about 1 Gy/day to about 5 Gy/day or about 1 Gy/day to about 3 Gy/day. Examples of suitable radiation therapy dosages include, but are not limited to about 1, 2, 3, 4, 5, 6, 7, and 8 Gy/day or a range between or including any of the individual values described herein. Specific dosages of an effective amount of radiation and/or drug-loaded micelle composition in the present methods may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the present technology.

In some embodiments, the present methods can include administering to lung, brain or esophageal cancer cells radiation therapy over a period of time of about 1 to about 5 days, overlapping with or in sequence with administration of a micelle composition. For example, radiation therapy may be administered to lung, brain or esophageal cancer cells from about 1 to about 5 days following pretreatment with a micelle composition including an effective amount of paclitaxel, 17-AAG, and/or rapamycin. Examples of suitable time periods for radiation therapy include, but are not limited to, about 1 day or about 1 to about 2, 3, 4, or about 5 days. The present methods can also include administering to lung, brain or esophageal cancer cells radiation therapy over a period of time simultaneously with or separately from administration of paclitaxel, 17-AAG, and/or rapamycin-loaded micelle. For example, radiation therapy can be administered to lung, brain or esophageal cancer cells at the same time as drug-loaded micelle.

In some embodiments, multiple rounds of radiation doses in conjunction with micelle compositions may be administered. For example, 2, 3, 4, 5, 6, 7, 8, 9, or even 10 rounds of the radiation/micelle composition doses may be administered, with days or weeks or even months in between each round. In some embodiments, the rounds of radiation/micelle compositions are administered 4, 5, 6, 7, 8, 9, or 10 days apart, and in others the rounds are administered 1, 2, 3, 4, 5, 6, 7, or 8 weeks apart, or even 1, 2, 3, 4, 5, or 6 months apart. Selecting the number of rounds and the interval(s) between each round is within the skill in the art and the bounds of routine experimentation and therefore, well within the scope of the present technology.

In some aspects, the present methods can include in vitro administration to lung, brain or esophageal cancer cells of an effective amount of radiation therapy in conjunction with the micelle compositions described herein. For example, the present methods can include administering to lung, brain or esophageal cancer cells harvested from laboratory culture, or any similar in vitro cell growth procedure known to a person having ordinary skill in the art, an effective amount of radiation therapy in conjunction with paclitaxel, 17-AAG, and/or rapamycin-loaded micelle. Additionally, an effective amount of radiation therapy can be administered in vitro to lung, brain or esophageal cancer cells sequentially, simultaneously, or separately at any of the dosage ranges described herein in conjunction with drug-loaded micelle.

In some aspects, the present methods include administering to lung, brain or esophageal cancer cells an effective amount of radiation therapy in conjunction with drug-loaded micelle composition in vivo. In some embodiments, the lung, brain or esophageal cancer cells are in a subject, such as a human or animal subject (e.g., mouse, rat, dog, cat, pig, horse, cow, monkey or the like). In certain embodiments, the micelle composition comprises an aqueous carrier and is administered to the subject intravenously or intraperitoneally.

The aqueous carrier may include saline or an aqueous carbohydrate solution. The aqueous carriers can include saline or an aqueous carbohydrate solution, for example, 0.9% NaCl solution or a 5% aqueous saccharide solution. The aqueous saccharide solution can be, for example, dextrose or glucose. The aqueous carrier can also be any sterile aqueous solutions of water-soluble salts, for example, NaCl. The aqueous solutions can also be isotonic. The aqueous solutions may be suitably buffered. Aqueous solutions, described herein, can be suitable for intravenous, intramuscular, subcutaneous, intraperitoneal, and intratumoral injection. Appropriate sterile aqueous media can be purchased or can be prepared by standard techniques well known to those skilled in the art.

In some aspects, the present methods include administering to a subject having lung, brain or esophageal cancer an effective amount of radiation therapy in conjunction with a PEG-b-PLA micelle composition that contains the drugs paclitaxel, 17-AAG, and/or rapamycin.

### Definitions

The following terms are used throughout as defined below.

The term “and/or” means any one of the items, any combination of the items, or all of the items with which this term is associated.

As used herein, singular articles such as “a”, “an”, and “one” are intended to refer to singular or plural. As such, this statement is intended to serve as antecedent basis for use of such exclusive terminology as “solely,” “only,” and the like in connection with the recitation of claim elements, or use of a “negative” limitation.

The term “about” can refer to a variation of up to ±10% of the value specified. For example, “about 50” can in some embodiments carry a variation from 45 to 55 percent. In other embodiments, “about” can refer to a variation of ±1%, ±2%, or ±5%. Unless indicated otherwise herein, the term “about” is intended to include values, e.g., weight percentages, proximate to the recited range that are equivalent in terms of the functionality of the individual ingredient, the composition, or the embodiment. In addition, unless indicated otherwise herein, a recited range (e.g., weight percentages) includes each specific value, integer, decimal, or identity within the range.

“Treating” or “treatment” within the context of the present technology, means an alleviation, in whole or in part, of symptoms associated with a disorder or disease, or slowing, or halting of further progression or worsening of those symptoms. As a non-limiting example of treatment, a subject can be successfully treated for lung cancer if, after receiving through administration an effective or therapeutically effective amount of one or more compounds or compositions described herein, the subject shows observable and/or measurable reduction in or absence of one or more signs and symptoms of lung cancer such as, but not limited to, reduced tumor size (e.g., reduced tumor volume), reduced morbidity and mortality, or improvement in quality of life relating thereto. Treatment, as defined herein, of a mammal, including a human being, is subject to medical aid with the object of improving the mammal's condition, directly or indirectly. Treatment typically refers to the administration of an effective amount of a drug-loaded micelle composition as described herein.

The “administration” of an effective amount of drug-loaded micelle composition includes, for example, parenteral or topical administration. Parenteral, for example, can be by infusion, injection, such as intravenous, and the patient can be a mammal, for example, a human. Parenteral or systemic administration includes, but is not limited to, subcutaneous, intravenous, intraperitoneal, and intramuscular injections. Injectable dosage forms or the present methods generally include, but are not limited to, aqueous suspensions, such as aqueous suspensions described herein. Topical administration generally includes, but is not limited to, powders, sprays, ointments, pastes, creams, lotions, gels, solutions, and patches. For example, aerosol sprays can be prepared using drug-loaded micelle, oleic acid, tricholoromonofluoromethane, dichlorodifluoromethane, dichlorotetrafluoroethane, and a standard, metered dose aerosol dispenser. Upon administration, the micelles can circulate intact, dissociate into individual polymer chains, release active agents (either by diffusion or micelle composition dissociation), distribute into tissue (e.g. tumors), and/or undergo renal clearance. The schedule of these events cannot be predicted with specificity, and these events significantly influence the anti-tumor activity of the active agents, such as paclitaxel, rapamycin, or 17-AAG.

“Effective amount” refers to the amount of a compound, composition or treatment (e.g., radiation therapy) required to produce a desired effect. Hence, a “therapeutically effective amount” of a compound or composition of the present technology in the context of treatment refers to an amount of the compound or composition that alleviates, in whole or in part, symptoms associated with a disorder or disease, or slows or halts further progression or worsening of those symptoms. In the context of prevention, a therapeutically effective amount prevents or provides prophylaxis for the disease or disorder in a subject at risk for developing the disease or disorder. Determining a therapeutically effective amount of a compound described herein for treating a particular disorder or disease is well within the skill in the art in view of the present disclosure. In the case of lung cancer, the effective amount of the compound or composition may reduce the number of lung cancer cells; reduce the tumor size; inhibit, to some extent, tumor growth; and/or relieve to some extent one or more of the symptoms associated with the disorder; inhibit (i.e., slow to some extent and preferably stop) cancer cell infiltration into peripheral organs; inhibit (i.e., slow to some extent and preferably stop) tumor metastasis. For the treatment of tumor dormancy or micrometastases, the therapeutically effective amount of the compound or composition may reduce the number or proliferation of micrometastases; reduce or prevent the growth of a dormant tumor; or reduce or prevent the recurrence of a tumor after treatment or removal (e.g., using an anti-cancer therapy such as surgery, radiation therapy, or chemotherapy). To the extent the compound or composition may prevent growth and/or kill existing cancer cells, it may be cytostatic and/or cytotoxic. For cancer therapy, efficacy in vivo can, for example, be measured by assessing the duration of survival, disease free survival (DFS), time to disease progression (TTP), duration of progression free survival (PFS), the response rates (RR), duration of response, time in remission, and/or quality of life. The effective amount may improve disease free survival (DFS), improve overall survival (OS), decrease likelihood of recurrence, extend time to recurrence, extend time to distant recurrence (i.e., recurrence outside of the primary site), cure cancer, improve symptoms of cancer (e.g., as gauged using a cancer specific survey), reduce appearance of second primary cancer, etc. In some embodiments, the effective amounts of radiation and one or more drug(s) as disclosed herein are synergistic, e.g., they have a more than additive effect or produce effects that cannot produced by either agent alone.

The terms “cancer” and “cancerous” refer to or describe the physiological condition in animal cells that is typically characterized by unregulated cell growth. Included in this definition are benign and malignant cancers as well as dormant tumors or micrometastases. Examples of cancer include but are not limited to, carcinoma, lymphoma, blastoma, sarcoma, and leukemia. More particular examples of such cancers include lung cancers such as small-cell lung cancer and non-small cell lung cancer, as well as brain and esophageal cancer.

The term “lung cancer” or “lung cancer cells” in the present methods includes, but is not limited to, the three main types and corresponding subtypes of lung cancer. Examples of types and subtypes of lung cancer include, but are not limited to, non-small cell lung cancer (including squamous cell carcinoma, adenocarcinoma, and large cell carcinoma), small cell lung cancer (including oat cell cancer), and lung carcinoid tumor.

The term “metastasis” refers to the spread of cancer from its primary site to other places in the body. Cancer cells can metastasize by breaking away from a primary tumor, penetrating into lymphatic and blood vessels, circulating through the lymphatic system or bloodstream, exiting the lymph or blood vessels and attaching and growing in normal tissues elsewhere in the body. Metastasis can be local or distant. At the new site, the cells reproduce, may establish a blood supply and can grow to form a secondary tumor, i.e., a metastatic tumor. Both stimulatory and inhibitory molecular pathways within the tumor cell regulate this behavior, and interactions between the tumor cell and host cells in the distant site are also significant.

The term “micrometastasis” refers to a small number of cells that have spread from the primary tumor to other parts of the body. Micrometastasis may or may not be detected in a screening or diagnostic test.

The term “tumor” refers to all neoplastic cell growth and proliferation, whether malignant or benign, and all pre-cancerous and cancerous cells and tissues.

The term “inhibit” or “inhibition” or “inhibiting,” in the context of neoplasia, tumor growth, or tumor or cancer cell growth. For example, inhibition may include, but is not limited to, delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition can be referred to as prevention, chemoprevention, tumor regression, or complete tumor regression. The inhibition can be about 10%, about 25%, about 50%, about 75%, or about 90% inhibition, with respect to progression that would occur in the absence of treatment.

The term “subject” or “patient” refers to a mammal, such as a cat, dog, rodent or primate. Typically the subject is a human, and, preferably, a human having or suspected of having a cancer such as lung cancer. The term “subject” and “patient” can be used interchangeably.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Paclitaxel (PTX or P) is a known chemotherapeutic agent. Paclitaxel derivatives that may be exchanged with paclitaxel in the formulations herein include docetaxel and other known derivatives.

17-Allylamino-17-demethoxy-geldanamycin (17-AAG or 17) is a derivative of the well-known natural product geldanamycin. Geldanamycin is obtainable by culturing the producing organism, Streptomyces hygroscopicus var. geldanus NRRL 3602. The compound 17-AAG is made semi-synthetically from geldanamycin, by reaction of geldanamycin with allylamine, described in U.S. Pat. No. 4,261,989 (Sasaki et al.), the disclosure of which is incorporated herein by reference.

Radiosensitizers are compounds or compositions that increase the sensitivity of cancer cells to radiation therapy.

Rapamycin (RAP or R), also known as sirolimus, is an immunosuppressant drug and a known mTor inhibitor. The mTOR complex 1 (mTORC1) drives cellular growth by controlling various processes that control protein synthesis and angiogenesis. Upstream signaling pathways of mTORC1 include the phosphatidylinositol 3-kinase (PI3K/Akt) pathway, which is frequently dysregulated in many cancers. For example, 60-70% of lung cancers have PI3K/Akt/mTORC1 pathway activation. Rapamycin derivatives that may be exchanged with rapamycin in the formulations herein include deforolimus, temsirolimus, everolimus, and CCI-779.

The term “micelle” refers to a particle of colloidal dimensions that exists in equilibrium with the molecules or ions in solution from it as formed. Generally, examples of micelles include, but are not limited to, lipid-like molecules that aggregate in aqueous solution to a spherical form. Micelle formation in solution occurs by a process called micellization, in which surface-active molecules or ions aggregate to form micelles. For example, micelles can be formed from amphiphilic molecules or polymers. The present methods include micelles, a core-shell structure formed from amphiphilic polymers that contains a lipophilic core that is capable of encapsulation. For example, poly(ethylene glycol)-block-poly(lactic acid) are a type of amphiphilic-block-copolymer that forms micelles.

Micelle compositions of the present technology are compositions that include micelles, as defined herein, loaded with drugs, e.g., paclictaxel, 17-AAG, and/or rapamycin. The compositions may include aqueous carriers, including water, saline, aqueous carbohydrate solutions and the like.

The terms “PEG-PLA” or “PEG-b-PLA” refers to poly(ethylene glycol)-block-poly(lactic acid). The poly(ethylene glycol) (PEG), or poly(ethylene oxide), block is a polymer (or oligomer) of ethylene oxide prepared by polymerization of ethylene oxide. PEG is commercially available at molecular weights of about 300 g/mol to about 10,000,000 g/mol. There are a number of uses of PEG, including commercial, chemical, and biological. One example use for PEG is in the research of chemical and biological agents, where PEG is used as the hydrophilic block of amphiphilic copolymers to create micelles, and other types of artificial vesicles. The poly(lactic acid) (PLA) block can include (D-lactic acid), (L-lactic acid), (D,L-lactic acid), or any combination thereof. PLA is a polyester of lactic acid having various well-known synthetic preparations. PLA often serves as the hydrophobic block of amphiphilic copolymers to create micelles, and other types of artificial vesicles. Various forms of PEG and PLA are available commercially, or they can be prepared according to methods well-known to those of skill in the art.

The term “drug loading” refers to the capacity for encapsulating the drugs and/or drug combinations of paclitaxel, 17-AAG, and/or rapamycin, or derivatives described herein, by micelle. Drug loading for multidrug micelles can approach, be equal to, or exceed the drug loading capacity of singe agent micelles.

The term “radiation therapy,” “radiation,” or “irradiation” in the present methods means the use of directed X-rays, gamma rays, beta rays, or charged particles to induce sufficient damage to a cell so as to limit its ability to function normally or to destroy the cell altogether. For example, high doses of X-rays can be used to treat cancer. Administration of radiation therapy can be external or internal. For example, external beam radiation therapy can be administered by a machine to aim high energy rays at cancer cells, such as a Precision X-RAD 320 (Precision X-Ray, North Branford, Conn.) to deliver X-ray radiation therapy (XRT). It will be appreciated that there will be many ways known in the art to determine the dosage and duration of treatment. Typical treatments are given as a one time administration and typical dosages range from about 1 to 200 units (Grays) per day. For example, a subject having lung cancer can be irradiated with about 1 to about 8 Gy/day over about 1 to about 5 days.

The terms “chemotherapeutic agent,” “chemotherapy agent,” “chemotherapy drug,” or “chemotherapeutic drug” refer to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include chemical compounds useful in the treatment of cancer. Examples of chemotherapeutic agents, but are not limited to, Taxol and paclitaxel.

The examples herein are provided to illustrate advantages of the present technology and to further assist a person of ordinary skill in the art with using the present technology. The examples should in no way be construed as limiting the scope of the present technology, as defined by the appended claims. The examples can include or incorporate any of the variations, aspects or aspects of the present technology as described herein. The variations, aspects or aspects described herein may also further include or incorporate variations of any or all other variations, aspects or aspects of the present technology.

### EXAMPLES

### Materials.

Materials for this general procedure include poly(ethylene glycol)-block-poly(D,L-lactic acid) (PEG-b-PLA) with molecular weight of poly(ethylene glycol) (PEG) of 4000 g/mol and molecular weight of poly(lactic acid) (PLA) of 2200 g/mol purchased from Advanced Polymer Materials Inc. (Montreal, CAN). The anticancer drugs paclitaxel (PTX), 17-allylamino-17-demethoxy-geldanamycin (17-AAG), and rapamycin (RAP) were purchased from LC Laboratories (Woburn, Mass.). A549 non-small cell lung cancer adenocarcinoma (A549 cells) were purchased from ATCC (Manassas, Va.). Crystal violet was purchased from Sigma-Aldrich (St. Louis, Mo.). All other reagents were obtained from Thermo Fisher Scientific Inc. (Fairlawn, N.J.) and were of analytical grade. Female athymic nude mice ages 6-8 weeks were purchased from Harlan Laboratories (Madison, Wis.).

### General Procedure.

### 1: Preparation and Characterization of Drug-Loaded PEG-b-PLA Micelles

One, two, or three of the anticancer drugs PTX, 17-AAG, and RAP with PEG-b-PLA were dissolved in a suitable water miscible solvent, such as tent-butyl alcohol or acetonitrile, and, if desired, with mixing and/or sonication. The solvent was removed, for example through lyophilization, to obtain drug-loaded micelles. Drug-loaded micelles can be rehydrated using an aqueous carrier, for example saline, and isolated, for example, by filtration and/or centrifugation.

Z-average diameter and polydispersity index (PDI) of drug-loaded PEG-b-PLA micelles at 25° C. were measured using a Zetasizer Nano-ZS (Malvern Instruments, UK) at a fixed angle of 173°. Prior to the measurement, drug-loaded PEG-b-PLA micelles were diluted 50 times with physiological saline.

The drug content of PEG-b-PLA micelles was quantified by a reverse-phase Shimadzu Prominence HPLC system (Shimadzu, Japan). Ten μL of drug-loaded PEG-b-PLA micelle solution were dissolved in 990 μL of acetonitrile. Ten μL of the dissolved solution were injected into a Zorbax RX-C8 analytical column (4.6 mm x250 mm, particle size 5 μm, Agilent) at a flow rate of 1.0 mL/min, a run time of 20 minutes, and a column oven temperature of 40° C. The separation of PTX, 17-AAG, and RAP was achieved with a mobile phase consisting of 55% of acetonitrile, and 45% of water containing 0.1% phosphoric acid and 1% methanol under isocratic conditions. PTX, 17-AAG, and RAP were detected at 227, 333, and 279 nm, respectively. Retention times of PTX, 17-AAG, and RAP were 3, 4, and 11 minutes, respectively.

### 2: A549 Cell Culture

A549 cells were cultured in RPMI 1640 medium supplemented with 10% fetal bovine serum and 1% penicillin/streptomycin. A549 cells were maintained at 37° C. under an atmosphere of 5% CO₂ in a humidified incubator.

### 3: In Vitro Clonogenic Assay

For the clonogenic assay procedure, a feeder layer of A549 cells was prepared by radiation at 30 Gy by using a 137Cs irradiator (JL Shepherd Model 109 Irradiator, JL Shepherd & Associates, CA). The feeder layer and non-irradiated A549 cells were seeded on 6-well plates to a total of 2500 cells/well. After seeding, cells were incubated overnight. Then, A549 cells were pretreated with one, two, or three drug-loaded micelle at 1-3 nM. Subsequently, cells were subjected to ionizing radiation (XRT) 0-8 Gy. Seven days after radiation treatment, the A549 cell culture medium was refreshed once and followed by incubation for 3 more days. After the incubation period, the medium was removed and, the A549 cells were washed with phosphate-buffered saline. A549 cells were then stained with a 0.5% crystal violet/methanol solution for 15 min at 37° C., and the number of colonies was counted using a cutoff of 50 aggregated cells. Plating efficiency (PE) was calculated as (colony count)/(inoculated non-irradiated cell number). Clonogenic survival of A549 cells was defined as (PE/PE of non-treatment). The survival fraction (SF) was calculated as (PE)/(PE at 0 Gy) of each treatment. A fitting curve was applied for the SF with a linear-quadratic equation: SF=exp(αD+βD²), where D is irradiation dose according to the procedure of Franken N A P, Rodermond H M, Stap J, Haveman J, Bree Cv. Clonogenic assay of cells in vitro. Nat Protoc. 2006;1(5):2315-9. doi:10.1038/nprot.2006.339. From the curve-fitting equation, the radiation dose necessary to kill 50% of cells in each treatment group was calculated to evaluate the sensitizer enhancement ratio (SER), defined as the radiation dose resulting in 50% cell kill without drug/radiation dose resulting in 50% cell kill with drug.

### 4: In Vivo Anticancer Efficacy of Drug-Loaded PEG-b-PLA Micelles With or Without Irradiation

For the in vivo drug-loaded micelle efficacy procedure, A549 cells were harvested from sub-confluent cultures after trypsinization. Mice were anesthetized with 1.5% isoflurane/oxygen; this state was maintained with 1% isoflurane/oxygen. The mice were then subcutaneously inoculated with A549 cells on the right side of the lower back (100 μL, 2×10⁶ cells/animal). Tumor volume was calculated as 0.5×a×b² with “a” as the larger diameter of the tumor and “b” as the smaller diameter of the tumor. After reaching a tumor volume of approximately 150 mm³, mice were randomized into 14 groups (n=4) as follows: 1. PTX at 60 mg/kg with radiation (PTX+XRT). 2. PTX at 60 mg/kg. 3. PTX/17-AAG (60/60 mg/kg) with radiation (P/17+XRT). 4. PTX/17-AAG (60/60 mg/kg) (P/17). 5. PTX/RAP (60/30 mg/kg) with radiation (P/R+XRT). 6. PTX/RAP (60/30 mg/kg) (P/R). 7. 17-AAG/RAP (60/30 mg/kg) with radiation (17/R+XRT). 8. 17-AAG/RAP (60/30 mg/kg) (17/R). 9. High dose Triolimus (PTX/17-AAG/RAP at 60/60/30 mg/kg) with radiation (High TRIO+XRT). 10. High dose Triolimus (PTX/17-AAG/RAP at 60/60/30 mg/kg) (High TRIO). 11. Intermediate dose Triolimus (PTX/17-AAG/RAP at 15/15/7.5 mg/kg) with radiation (Int. TRIO+XRT). 12. Intermediate dose Triolimus (PTX/17-AAG/RAP at 15/15/7.5 mg/kg) (Int. TRIO). 13. Vehicle (empty PEG-b-PLA micelle at 105 mg/kg) with XRT (Vehicle+XRT). 14. Vehicle (empty PEG-b-PLA micelle at 105 mg/kg) (Vehicle). For each dose, 200 4/20 g mice body weight was intravenously administered to anesthetized nude mice. For groups having combined drug-loaded micelle and radiation treatment, A549 cell inoculated mice (A549 mice) were treated with drug-loaded PEG-b-PLA micelles prior to radiation, and then the A549 mice were subjected to 5 days of 3 Gy/day radiation. Body weights of A549 mice and diameters of tumors were recorded by a portable scale and a digital caliper (Fisher Scientific, Pittsburgh, Pa.), respectively. All mice used for this study were euthanized either when tumor volume reached 400% from initial tumor volume or on day 85. All animal experiments were approved by UW-Madison's Institutional Animal Care and Use Committee and conducted in accordance with institutional and NIH guidance.

### 5: Hematological, Histologic, and Immunohistochemical Analysis

PTX (60 mg/kg), P/17 (60/60 mg/kg), P/R (60/30 mg/kg), 17/R (60/30 mg/kg), High TRIO (PTX/17-AAG/RAP: 60/60/30 mg/kg), Int. TRIO (PTX/17-AAG/RAP: 12/12/6 mg/kg), Low TRIO (PTX/17-AAG/RAP: 2.4/2.4/1.2 mg/kg), and vehicle control (PEG-PLA only:105 mg/kg) were intravenously injected into A549 tumor-bearing female athymic nude mice. Animals were euthanized 24 hours post-injection. For the evaluation of Triolimus followed by XRT treatment group, High TRIO was intravenously injected on day 1, followed by fractionated radiation of 3 Gy for five days. At day six, animals were euthanized (TRIO+XRT group). The control group received a vehicle injection followed by XRT treatment (Vehicle+XRT group).

Whole blood was collected in both lithium heparin and K₂EDTA coated tubes just before euthanization. Whole blood in lithium heparin coated tubes was centrifuged at 3,000 rpm for 5 min to separate plasma for comprehensive metabolic panel. Whole blood in a K₂EDTA coated tube was used for complete blood count (CBC). CBC and comprehensive metabolic panels were conducted by the Clinical Pathology Laboratory of School of Veterinary Medicine at the University of Wisconsin, Madison, Wis.

Tumor and normal tissue were fixed for 24 hours in 10% neutral buffered formalin, dehydrated through graded ethyl alcohols, paraffin-embedded, cut at 5 μm, and mounted on charged glass slides. H&E stained sections of the tumor and normal tissue including heart, lung, liver, kidney, spleen, and brain were prepared according to standard procedure. Tissue necrosis was assessed by light microscopy as percent total tumor area. Normal tissue sections were assessed for morphologic evidence of organ toxicity by an observer blinded to treatment. Immunostaining for Ki-67, cleaved caspase-3, Akt, phospho-Akt, and Hsp70 was carried out on a Roche Ventana Medical System Discovery XT Automated platform using proprietary Ventana-Roche reagents except for DaVinci Antibody Diluent and Cat-Hematoxylin (Biocare Medical). The slides were deparaffinized, and heat-induced epitope retrieval was performed in the form of “cell conditioning” using CCI Buffer (EDTA buffer) for approximately 44 minutes. For Ki-67 detection, slides were then incubated with anti-human Ki-67 antibody (rabbit monoclonal, US Biological), 1:100, 28 minutes at 37° C., rinsed, and incubated with OmniMap anti-rabbit HRP for 12 minutes. For cleaved caspase-3, slides were incubated with anti-human cleaved caspase-3 antibody (rabbit polyclonal, Cell Signaling Technology), 1:400, 28 minutes at 37° C., rinsed and incubated with OmniMap anti-rabbit HRP for 12 minutes. For Akt, slides were incubated with anti-human Akt antibody (rabbit monoclonal, Cell Signaling Technology), 1:400, 16 minutes at 37° C., rinsed and incubated with OmniMap anti-rabbit HRP for 8 minutes. For phospho-Akt, slides were incubated with anti-human phospho-Akt antibody (rabbit monoclonal, Cell Signaling Technology), 1:50, 4 hour at 20° C., rinsed and incubated with UltraMap anti-rabbit HRP for 12 minutes. For HSP70, slides were incubated with HSP70 antibody (mouse monoclonal, Abcam), 1:3200, 28 minutes at 37° C., rinsed and incubated with UltraMap anti-mouse HRP for 12 minutes. The slides were then rinsed, incubated with ChromoDAB detection reagent, rinsed again and counterstained with Biocare Medical's Cat-Hematoxylin diluted 1:5 in distilled water. Immunohistochemical staining was assessed as follows: for Ki-67 enumeration, four representative 400× fields per replicate tumor were quantified using ImmunoRatio software [22]; the average of four measurements per replicate was taken. For cleaved caspase, positive cells were enumerated manually in four representative 400× viable tumor fields using light microscopy, and an average of four measurements per replicate was taken. Cytoplasmic Akt, pAKT and HSP70 immunoreactivity were assessed semi-quantitatively by light microscopy as negative (<1% cells positive), focal (1-25% cells positive) or diffuse (>25% cells positive) and weak, moderate or strong based on the intensity of staining. All morphologic and immunohistochemical assessments were performed by a pathologist blinded to treatment groups.

### 6: Statistical Analysis

Statistical analysis was performed using Student's t-tests. Differences were deemed statistically significant if the two-tailed p-value was less than 0.05.

### Example 1

### Preparation and Characterization of Paclitaxel, 17-AAG, and/or Rapamycin Loaded PEG-b-PLA Micelles

Drug-loaded PEG-b-PLA micelles were prepared by a freeze-drying method described above in General Procedure 1. Briefly, 6 mg of PTX and 17-AAG, 3 mg of RAP, and 105 mg of PEG-b-PLA were dissolved in 1 mL of pre-heated tent-butyl alcohol at 60° C. in a cylindrical glass tube, followed by a rapid addition of 1 mL of pre-heated distilled water at 60° C. with vigorous vortex. Subsequently, the mixed solution was frozen in dry-ice, and lyophilized for 3 days at 100 μBar (9.87×10⁴ atm), −20° C. by a freeze-drying instrument. One drug (PTX only) and two drug (PTX/17-AAG, PTX/RAP, and 17-AAG/RAP) loaded PEG-b-PLA micelles were similarly prepared. The freeze-dried samples were kept in a freezer until use. At the point of use, 1.0 mL of physiological saline was added to rehydrate drug-loaded PEG-b-PLA micelles, followed by centrifugation at 13,000 rpm for 5 min and filtration through a 0.2 μm syringe filter to remove residue and sterilize. Table 1 shows the size, polydispersity (PDI) and drug loading of the micelles. PEG-b-PLA micelles solubilizing PTX alone or multiple drugs had an average Z-diameter of ca. 35 nm with a PDI of 0.12, irrespective of contents. Drug loading slightly increased particle size and decreased PDI. All drugs loaded in PEG-b-PLA micelles had high aqueous solubility, even multi-drug formulations. The PTX alone-loaded micelles precipitated 6 hours after rehydration in physiological saline, however, Triolimus and dual drug-loaded micelles did not precipitate at room temperature up to 24 hours after reconstitution.

TABLE 1
Size and PDI of drug-loaded PEG-b-PLA micelles

| | Particle size [nm] | PDI | Loading [mg/mL] | |
|---|---|---|---|---|
| Triolimus | 34 ± 1 | 0.12 ± 0.01 | PTX | 6.4 ± 0.1 |
| | | | 17-AAG | 6.3 ± 0.2 |
| | | | RAP | 3.7 ± 0.1 |
| PTX/17- | 33 ± 1 | 0.11 ± 0.01 | PTX | 6.4 ± 0.3 |
| AAG | | | 17-AAG | 6.2 ± 0.3 |
| PTX/RAP | 35.1 ± 0.4 | 0.118 ± 0.003 | PTX | 6.2 ± 0.2 |
| | | | RAP | 3.6 ± 0.3 |
| 17-AAG/ | 35.1 ± 0.3 | 0.11 ± 0.01 | 17-AAG | 5.8 ± 0.2 |
| RAP | | | RAP | 3.2 ± 0.1 |
| PTX | 34 ± 1 | 0.109 ± 0.005 | PTX | 6.4 ± 0.3 |
| Vehicle | 33.9 ± 0.5 | 0.131 ± 0.002 | | |

### Example 2

### Efficacy of One, Two, and Three Drug-Loaded Micelles with Radiation for A549 Cells In Vitro

Following General Procedure 3, A549 cells were pretreated with drug-loaded micelles PTX/17-AAG/RAP (Triolimus) at 1 to 3 nM or PTX, PTX/17-AAG, PTX/RAP, or 17-AAG/RAP at 2 nM. Characterization was performed as described above.

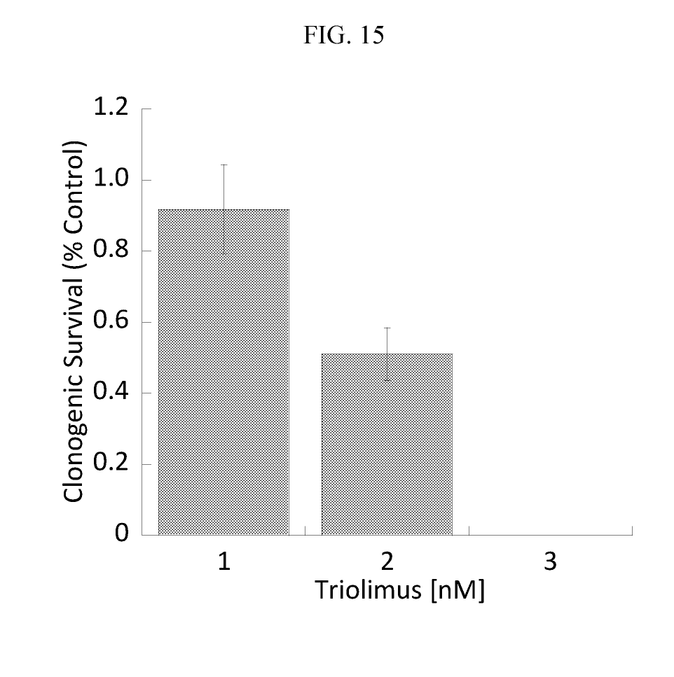

The effect Triolimus on clonogenic survival of A549 cells is shown in FIG. 15. The inhibitory effect of Triolimus was dose-dependent (1 nM: 0.92±0.13, 2 nM: 0.51±0.07), and A549 cells did not survive after exposure to 3 nM of Triolimus. Triolimus effectively inhibited clonogenic survival, even at concentrations as low as 2 nM.

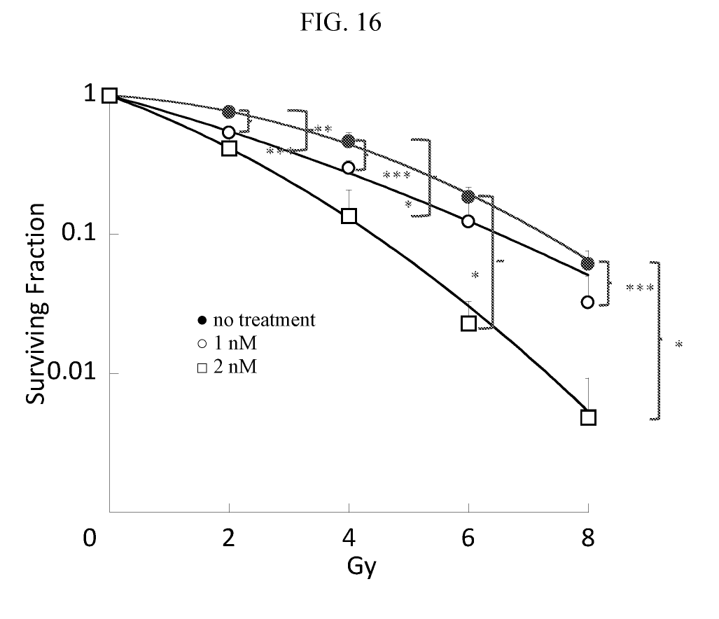

The effect of Triolimus on radiosensitization is shown in FIG. 16. Similar to clonogenic survival results, the radiosensitizing effect of Triolimus was dose-dependent. Interestingly, Triolimus had a significant radiosensitizing effect as low as 1 nM when combined with 2 Gy of radiation. This effect was augmented at 2 nM (the SER value at 1 and 2 nM: 1.58 and 2.23, respectively). These results suggest that Triolimus is a potent radiosensitizer.

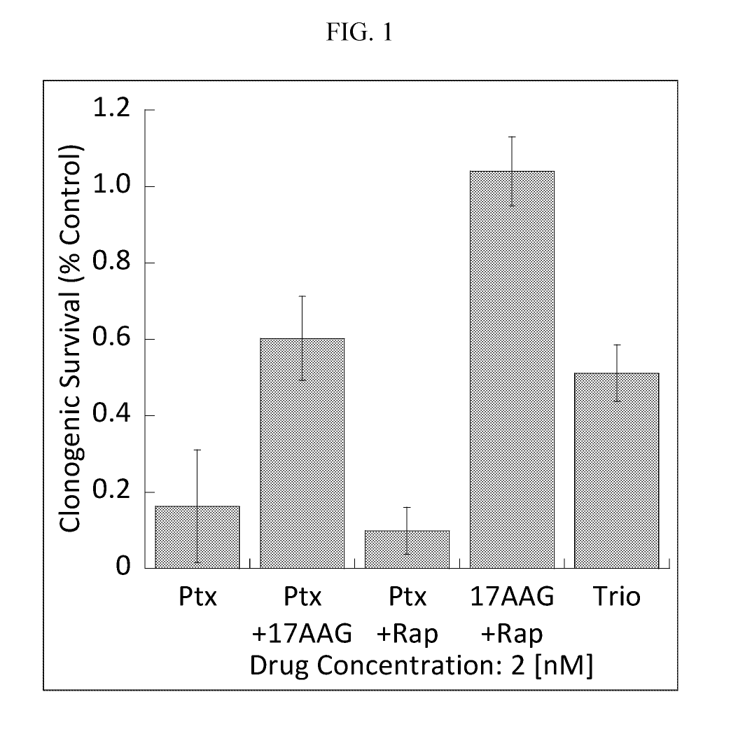

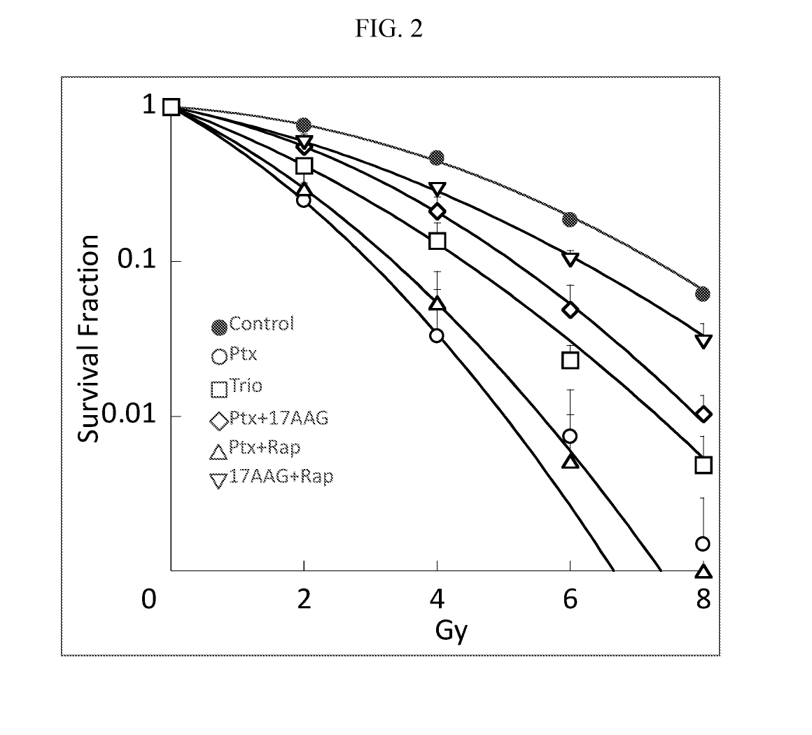

A549 cells treated with drug-loaded micelles with radiation (XRT) showed effective anticancer activity for one, two, and three drug-loaded micelles. The effects of drug combinations on clonogenic survival of A549 cells were evaluated by using single (PTX) and dual P/17, P/R, and 17/R loaded PEG-b-PLA micelles as well as TRIO at a fixed concentration of 2 nM (FIG. 1). Interestingly, the most effective drug combination that inhibited clonogenic survival was P/R, followed by PTX, TRIO, P/17, and 17/R with the following clonogenic survival rate: 0.10±0.06, 0.16±0.15, 0.51±0.07, 0.60±0.11, and 1.04±0.09, respectively. A similar trend was observed in radiosensitization studies (FIG. 2). A549 cells were especially susceptible to P/R treatment with 8 Gy of ionizing radiation. However, the SER value revealed that the strongest radiosensitizing effect was obtained by treatment with PTX (3.34), followed by P/R (2.95), TRIO (2.23), P/17 (1.62), and 17/R (1.44) (Table 2). Interestingly, the SER value for the combination of P/R was more than 2-times than P/17, despite the similar molar ratio of RAP in each formulation suggesting the radiosensitization of PTX is higher than 17-AAG. Comparing Triolimus and P/17, the addition of RAP to PTX and 17-AAG in Triolimus greatly enhanced its radiosensitizing potency despite a slight decrease in PTX and 17-AAG molar ratios. In Triolimus, the radiosensitizing effects of these three drugs appear to achieve synergy in vitro.

TABLE 2
Molar ratio of drugs at a concentration of
2 nM and the SER values of each micelle

[nM]

| Drug | PTX | 17-AAG | RAP | SER |
|---|---|---|---|---|
| Triolimus | 0.7 | 1.0 | 0.3 | 2.23 |
| PTX | 2.0 | | | 3.34 |
| PTX/17AAG | 0.8 | 1.2 | | 1.62 |
| PTX/RAP | 1.4 | | 0.6 | 2.95 |
| 17AAG/RAP | | 1.5 | 0.5 | 1.44 |

### Examples 3-8

### Efficacy of One, Two, and Three Drug-Loaded Micelles With or Without Radiation for A549 Cells In Vivo

Examples 3-8 all follow General Procedure 4 for in vivo drug-loaded micelle efficacy, mice were subcutaneously inoculated with A549 cells on the right side of the lower back (100 μL, 2×10⁶ cells/animal). Mice inoculated with A549 cells (A549 mice) with a tumor size of 150 mm³ were dosed with 200 4/20 g mice body weight intravenously with 60 mg/kg PTX-loaded micelle (Example 3), 60/60/30 mg/kg PTX/17-AAG/RAP-loaded micelle (“High TRIO”) (Example 4), 15/15/7.5 mg/kg PTX/17-AAG/RAP-loaded micelle (“Int. TRIO”) (Example 5), 60/60 mg/kg PTX/17-AAG-loaded micelle (Example 6), 60/30 mg/kg PTX/RAP-loaded micelle (Example 7), and 60/30 mg/kg 17-AAG/RAP-loaded micelle (Example 8). A549 mice were also treated with 200 4/20 g mice body weight with a non-drug loaded micelle (vehicle; only PEG-b-PLA micelle at 105 mg/kg). Following General Procedure 4, A549 mice treated with the various drug-loaded micelles or vehicle were also subjected to 5 days of 3 Gy/day radiation (XRT). Characterization of A549 mice treated with the various drug loaded micelles with XRT, the various drug loaded micelles only, vehicle with XRT, or vehicle only was performed following General Procedure 4.

### Example 3

### Efficacy of Paclitaxel-Loaded Micelle With and Without Radiation for A549 Cells In Vivo—(60 mg/kg PTX-Loaded Micelle)

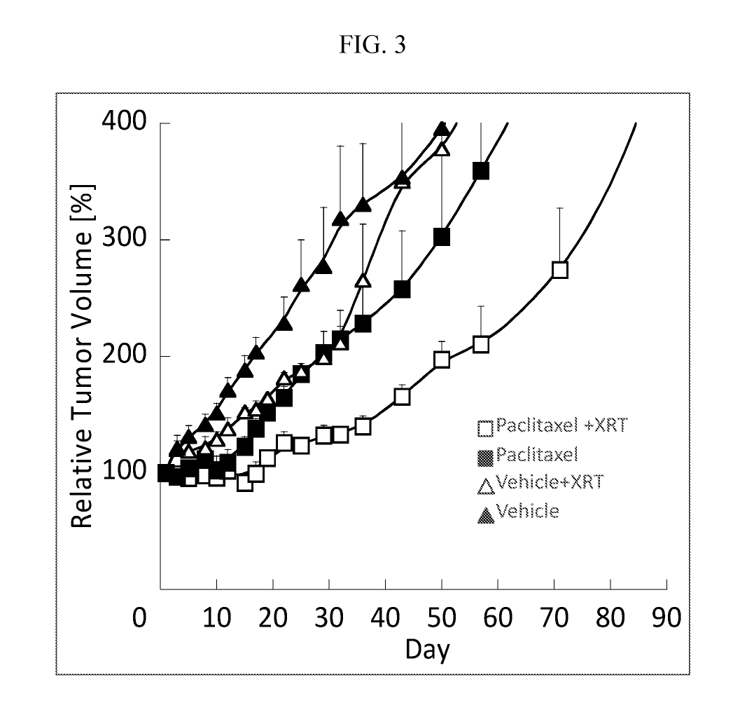

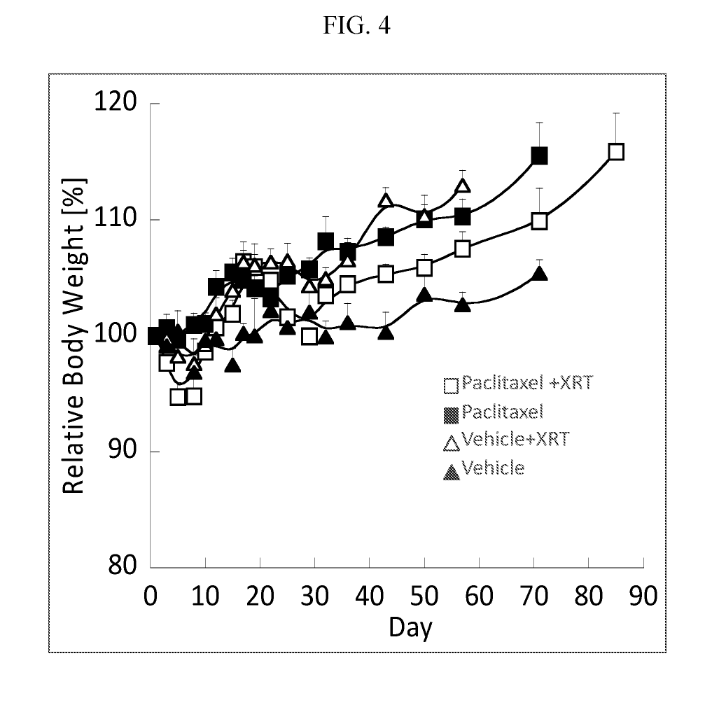

FIG. 3 shows the rate of A549 cells growth rate as a function of time (days). Compared to the vehicle control group, radiation alone and PTX alone slightly inhibited tumor growth. A549 mice treated with PTX-loaded micelle with XRT showed a modest reduction in tumor growth rate. Body weights of all A549 mice studied were recorded following General Procedure 4 and plotted as a function of time (days), as shown in FIG. 4. Body weights for A549 mice treated with PTX-loaded micelle with XRT, PTX-loaded micelle only, vehicle with XRT, and vehicle only showed little effect on the rate of relative body weight growth. Decreases in body weight for A549 mice treated with drug-loaded micelle correlates to a high drug treatment method toxicity. The moderate growth in relative body weight for the PTX-loaded micelle treated A549 mice, shown in FIG. 4, suggests a low drug toxicity with or without XRT.

### Example 4

### Efficacy of Paclitaxel/17-AAG/Rapamycin Loaded Micelle at High Dosage with and Without Radiation for A549 Cells In Vivo—60/60/30 mg/kg PTX/17-AAG/RAP Loaded Micelle (High TRIO)

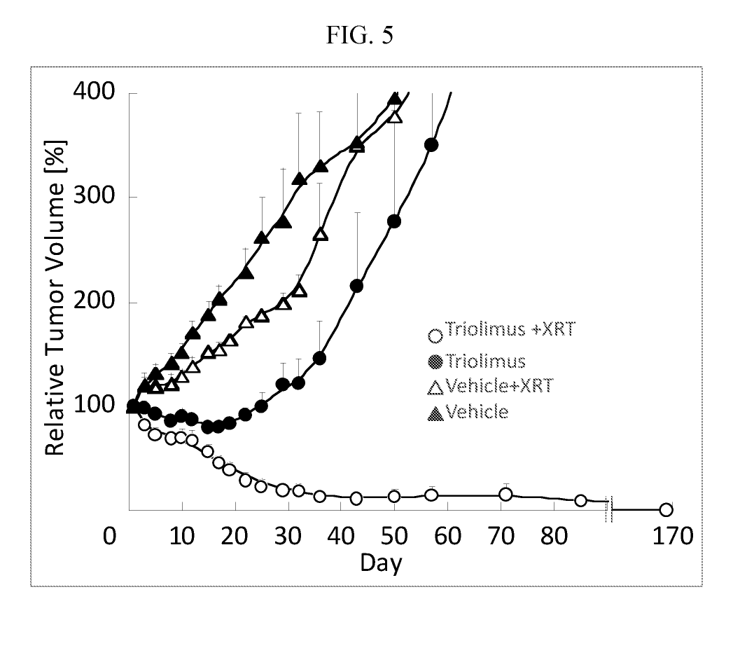

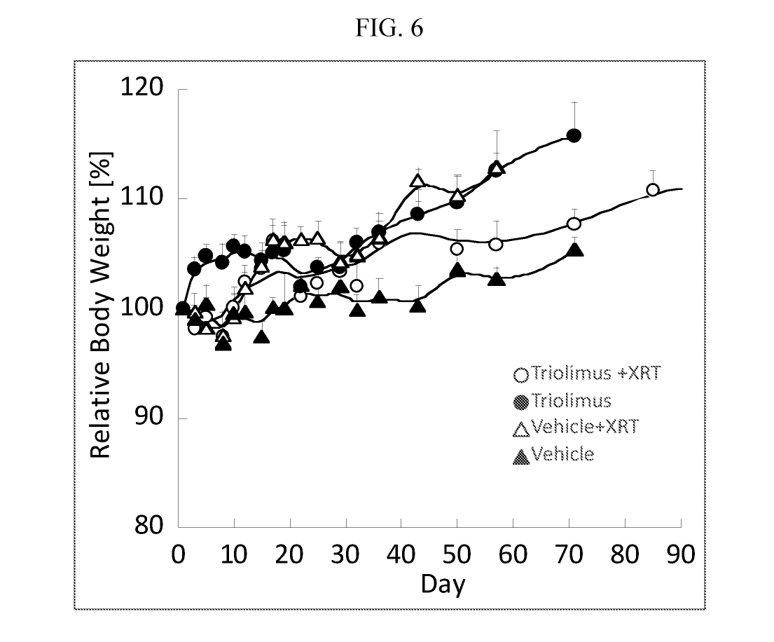

FIG. 5 shows that A549 mice treated with High TRIO with XRT had a drastic reduction in tumor growth rate over 90 days and complete tumor regression after 170 days. However, A549 mice treated with High TRIO only or vehicle with XRT showed just a modest reduction in tumor growth rate compared to the vehicle only tumor growth rate. Results for A549 mice treated with High TRIO with XRT were unexpected compared to A549 mice treated with PTX-loaded micelle with XRT, discussed in Example 3, and in vitro A549 lung cancer cell anticancer effects, discussed in Example 2. This tumor control lasted for up to 24 weeks, during which time no local recurrences were observed. Thus, High TRIO was confirmed to be a potent radiosensitizer in vivo. Additionally, FIG. 6 shows low drug toxicity in A549 mice treated with High TRIO with XRT, High TRIO only, vehicle with XRT, or vehicle only. FIG. 6 shows growth rate in A549 mice relative body weight measured as a function of time for all High TRIO or vehicle treated A549 mice.

### Example 5

### Efficacy of Paclitaxel/17-AAG/Rapamycin Loaded Micelle at Intermediate Dosage With and Without Radiation for A549 Cells In Vivo—15/15/7.5 mg/kg PTX/17-AAG/RAP Loaded Micelle (Int. TRIO)

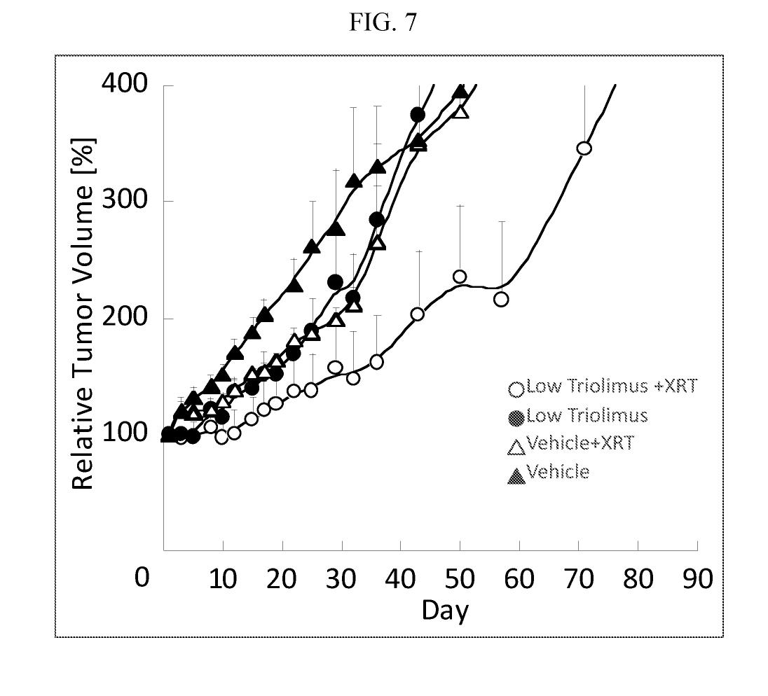

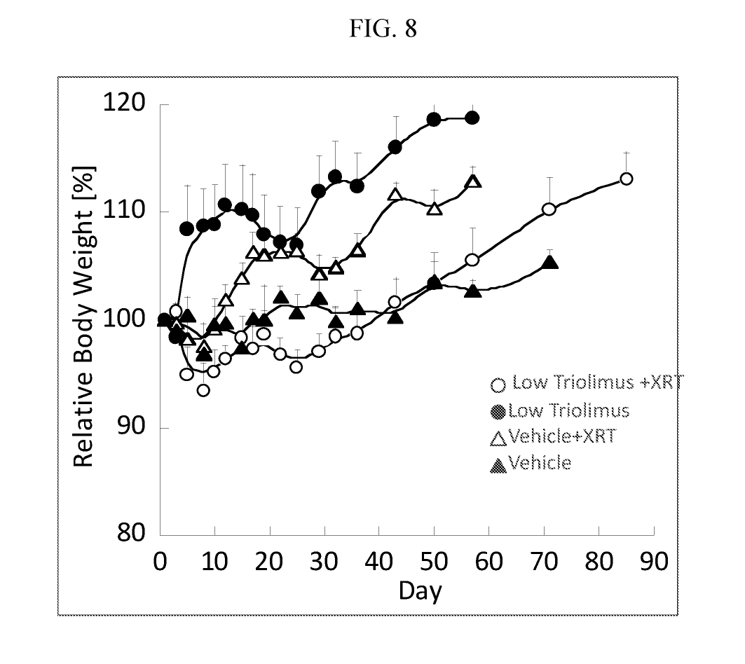

FIG. 7 shows that A549 mice treated with Int. TRIO with XRT had a modest reduction in tumor growth rate over 90 days compared to the vehicle only tumor growth rate. A549 mice treated with Int. TRIO only or vehicle with XRT showed little reduction in tumor growth rate compared to the vehicle only tumor growth rate. Additionally, FIG. 8 shows low drug toxicity in A549 mice treated with Int. TRIO with XRT, Int. TRIO only, vehicle with XRT, or vehicle only. FIG. 8 shows growth rate in A549 mice relative body weight measured as a function of time (days) for all Int. TRIO or vehicle treated A549 mice, where there was no significant reduction in the relative body weight growth rate.

### Example 6

### Efficacy of Paclitaxel/17-AAG-Loaded Micelle With and Without Radiation for A549 Cells In Vivo—60/60 mg/kg PTX/17-AAG-Loaded Micelle (P/17)

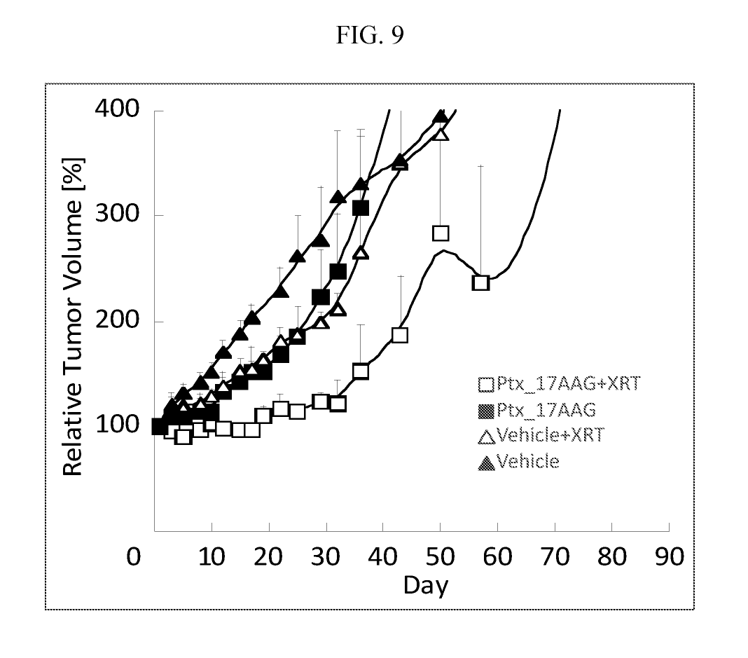

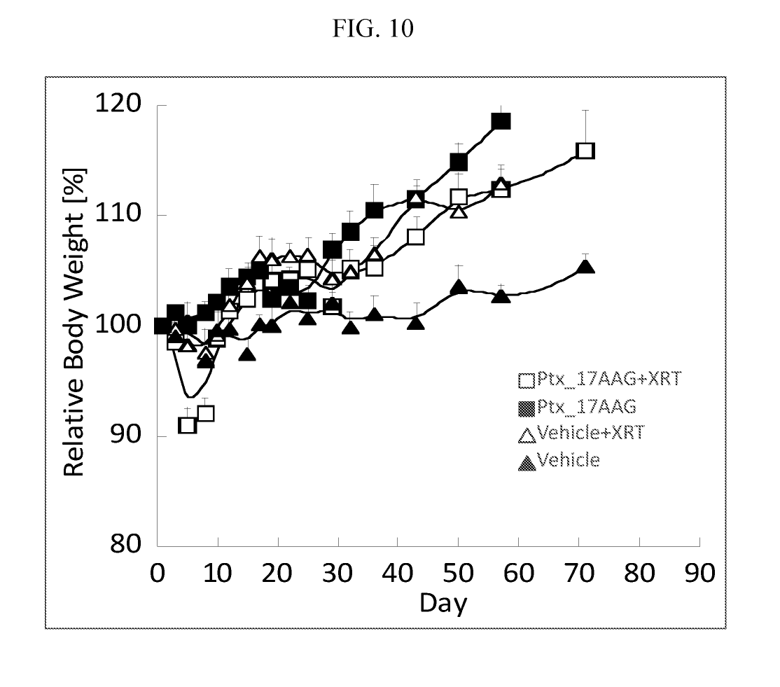

FIG. 9 shows A549 mice treated with PTX/17-AAG-loaded micelle with XRT (P/17+XRT) had a slight reduction in tumor growth rate over 90 days, however, its radiosensitizing effect was less potent than the combination of PTX and radiation. A549 mice treated with P/17 only or vehicle with XRT showed little reduction in tumor growth rate compared to the vehicle only tumor growth rate. Additionally, FIG. 10 shows low drug toxicity in A549 mice treated with P/17+XRT, P/17 only, vehicle with XRT, or vehicle only. FIG. 10 shows the growth rate in A549 mice relative body weight measured as a function of time (days) for all P/17 or vehicle treated A549 mice, where there was no significant reduction in the relative body weight growth rate.

### Example 7

### Efficacy of Paclitaxel/Rapamycin-Loaded Micelle With and Without Radiation for A549 Cells In Vivo—60/30 mg/kg PTX/RAP-Loaded Micelle (P/R)

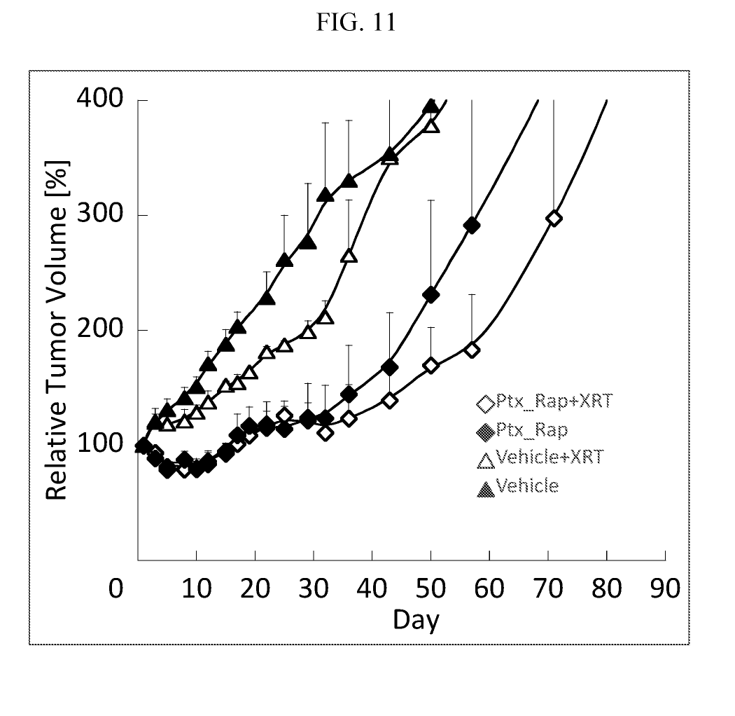

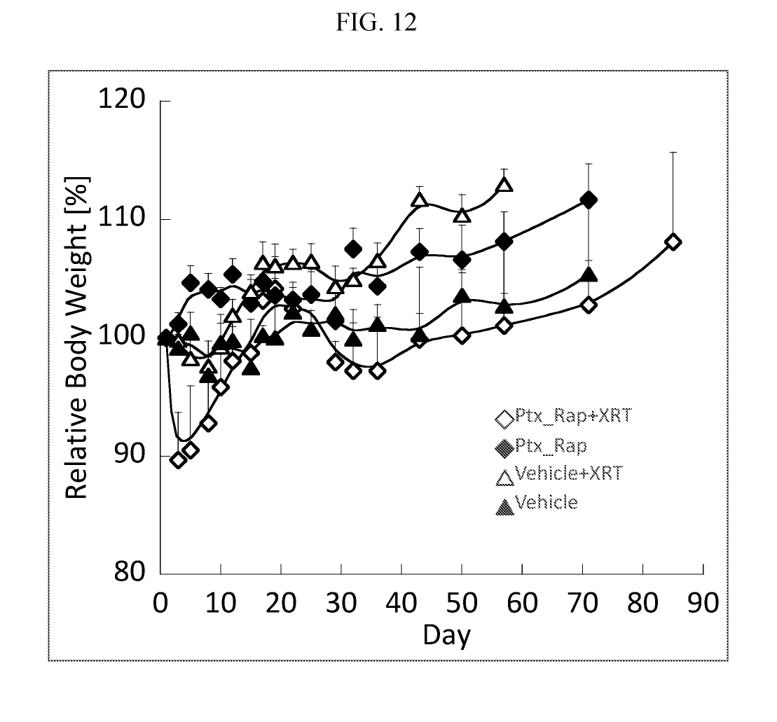

FIG. 11 shows A549 mice treated with P/R with XRT (P/R+XRT) or P/R only had a significant reduction in tumor growth rate in the first 30 days compared to the vehicle only tumor growth rate. A549 mice treated with vehicle with XRT showed little reduction in tumor growth rate compared to the vehicle only tumor growth rate. P/R+XRT was also more potent than P/17+XRT. However, compared to PTX+XRT, P/R+XRT did not prolong the tumor growth delay. Additionally, FIG. 12 shows low drug toxicity in A549 mice treated with P/R+XRT, P/R only, vehicle with XRT, or vehicle only. FIG. 12 shows the growth rate for A549 mice relative body weight measured as a function of time (days) for all P/R-loaded micelle or vehicle treated A549 mice, where there was no significant reduction in relative body weight growth rate.

### Example 8

### Efficacy of 17-AAG/Rapamycin Loaded Micelle With and Without Radiation for A549 Cells In Vivo—60/30 mg/kg 17-AAG/RAP-Loaded Micelle (17/R)

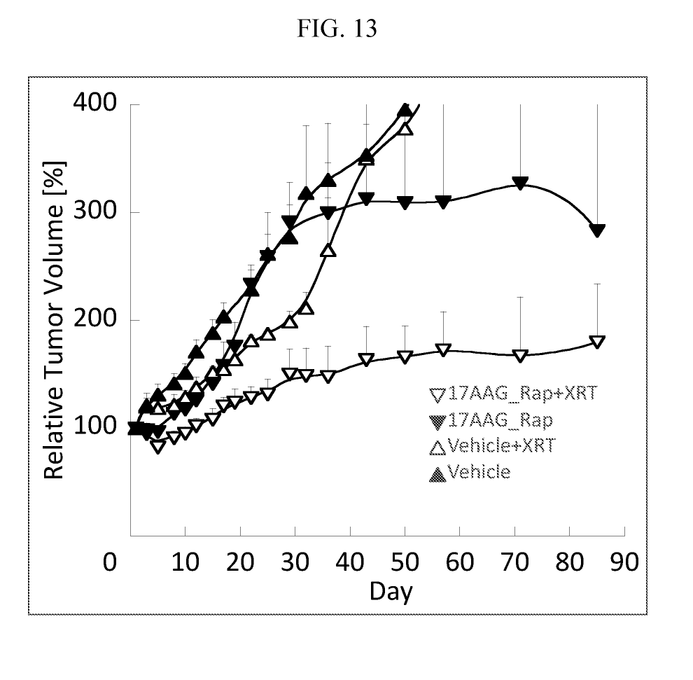

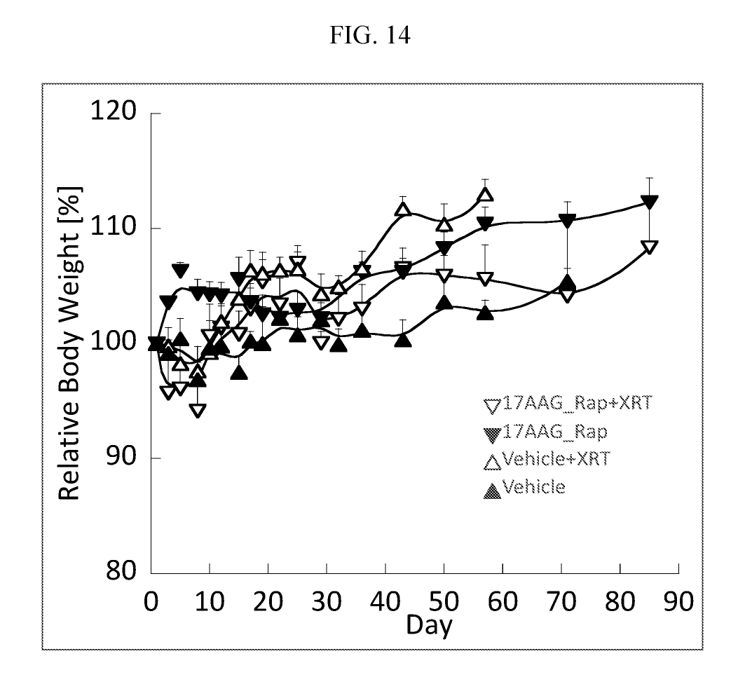

FIG. 13 shows A549 mice treated with 17-AAG/RAP-loaded micelle with XRT (17/R+XRT) dramatically inhibited tumor growth, despite in vitro study results showing less effective radiosensitization than other single, dual and Triolimus treatment (FIG. 2, Table 2). A549 mice treated with 17-AAG/RAP-loaded micelle only or vehicle with XRT showed no significant reduction in tumor growth rate compared to the vehicle only tumor growth rate. Additionally, FIG. 14 shows low drug toxicity in A549 mice treated with 17/R+XRT, 17/R only, vehicle with XRT, and vehicle only. FIG. 14 shows the growth rate for A549 mice relative body weight measured as a function of time (days) for all 17-AAG/RAP-loaded micelle or vehicle treated A549 mice, where there was no significant reduction in relative body weight growth rate.

### Example 9

### Histologic and Immunohistochemical Analysis of Tumor Growth

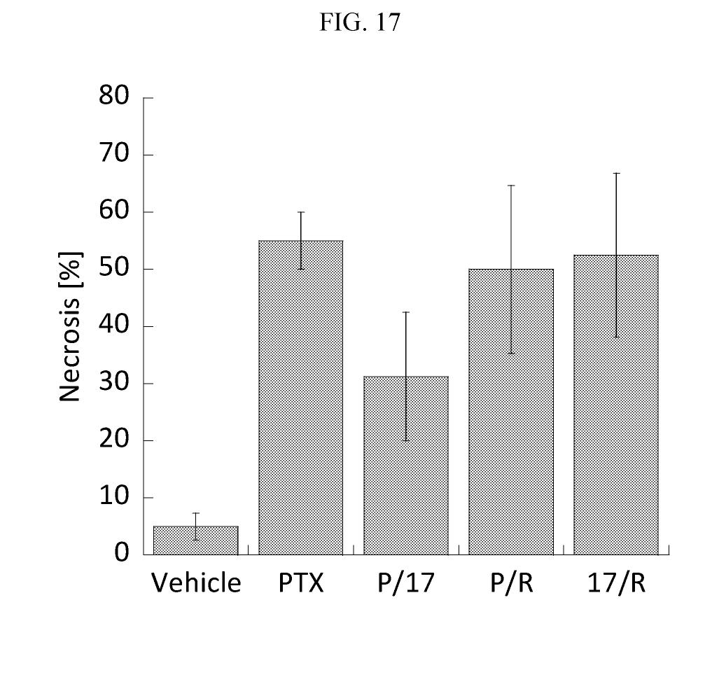

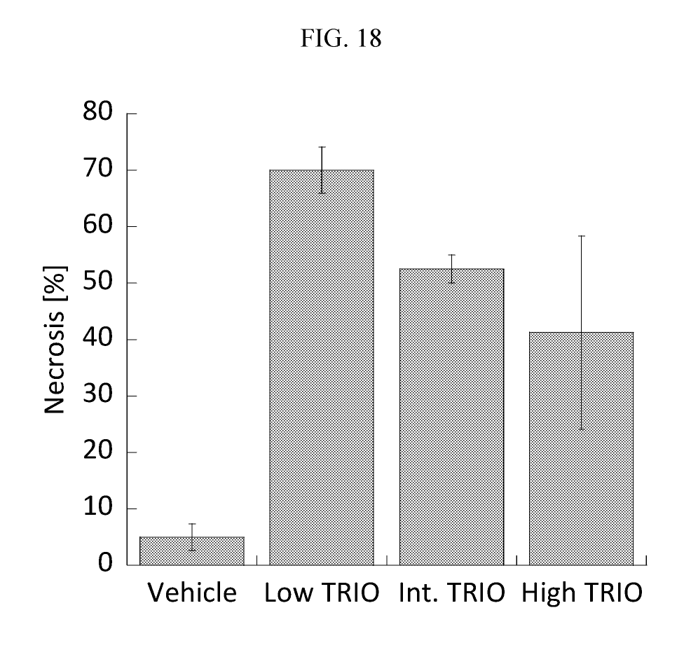

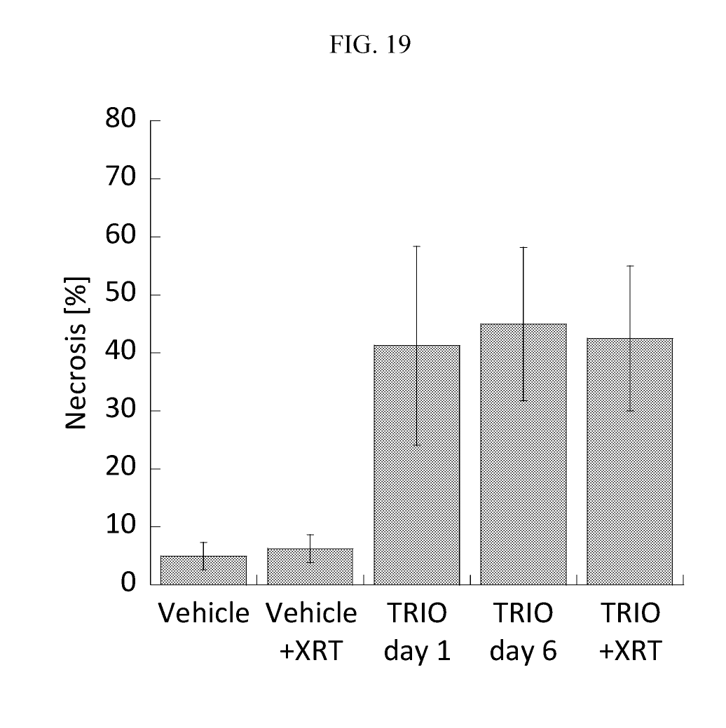

Following General Procedure 5, histologic and immunohistochemical analysis of tumor growth was performed. The percent of tumor necrosis is shown in FIGS. 17-19. For single and dual drug treated groups, the amount of necrosis was similar between PTX, P/R, and 17/R. Compared to those three treatments, relatively less necrosis was observed in the P/17 treated group (FIG. 17). Percent necrosis decreased with increasing drug concentrations in the Triolimus group (TRIO) (FIG. 18). The addition of radiation to TRIO (TRIO+XRT) did not increase the amount of necrosis (FIG. 19). Necrosis was augmented in the Triolimus treatment group and was sustained for 6 days. The level of necrosis was similar between TRIO day 6 and TRIO+XRT, suggesting that the induction of necrosis is caused by Triolimus administration.

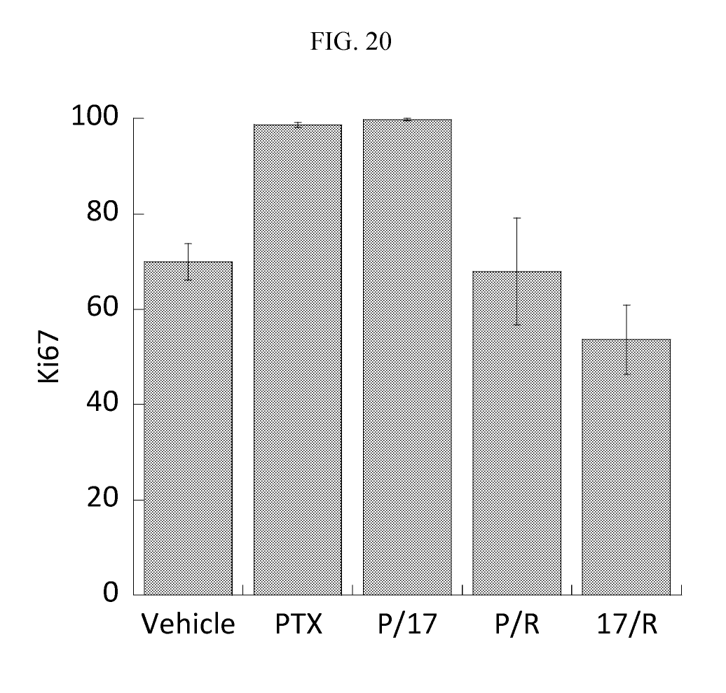

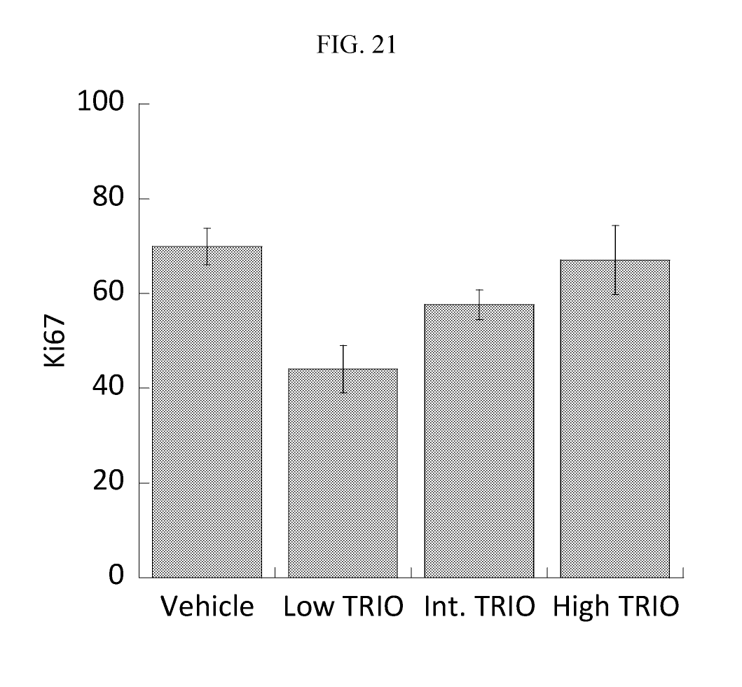

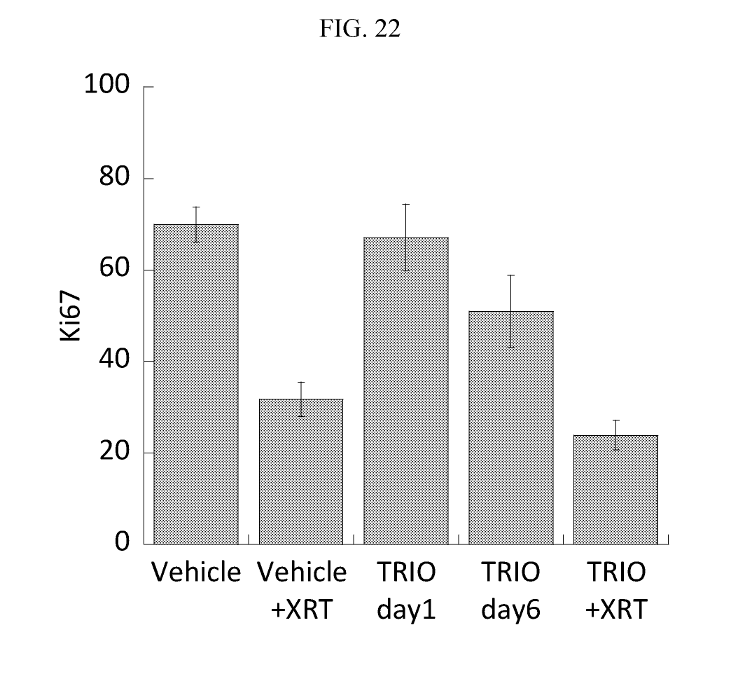

Ki-67 proliferative index of single drug, dual drug, and Triolimus treatment groups are shown in FIGS. 20-22. PTX and P/17 treatments enhanced tumor proliferation (FIG. 20). Both P/R and 17/R treatment led to Ki-67 indices similar to vehicle treatment (FIG. 20). Results from the Triolimus treatment group indicate that lower drug concentrations exert a more effective antiproliferative effect (FIG. 21). A dramatic reduction in Ki-67 index was observed from the vehicle+XRT treated group (FIG. 22). TRIO group did not induce antiproliferative effect on day one; however, Ki-67 values on day six were relatively low (FIG. 22). Thus, it appears that the antiproliferative effects of high dose Triolimus (High TRIO) treatment are not immediate but occur over time. The lowest Ki-67 values were observed in the TRIO+XRT group (FIG. 22) indicating that the combination of Triolimus and radiation potently inhibits tumor cell proliferation.

Not wishing to be bound by theory, these observations suggest the combination of Triolimus and radiation appears to result in the favorable tumor necrosis observed with drug treatment alone and the favorable anti-proliferative effects of radiation alone. This pro-therapeutic duality may underpin that observed in vivo efficacy of High TRIO plus radiation.

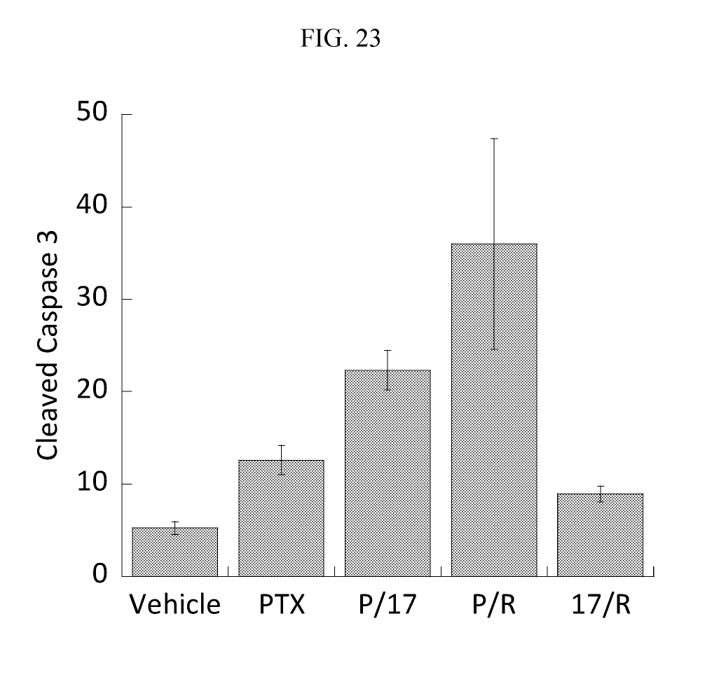

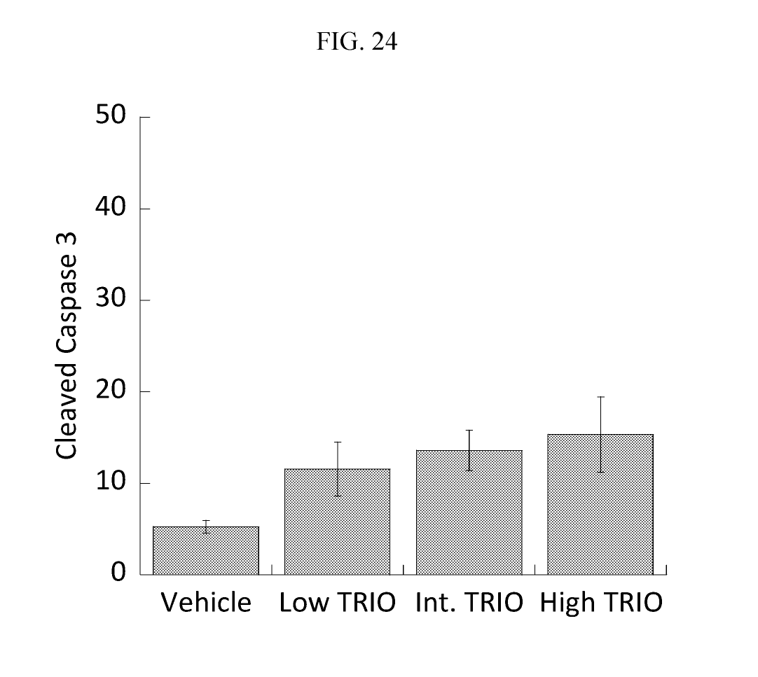

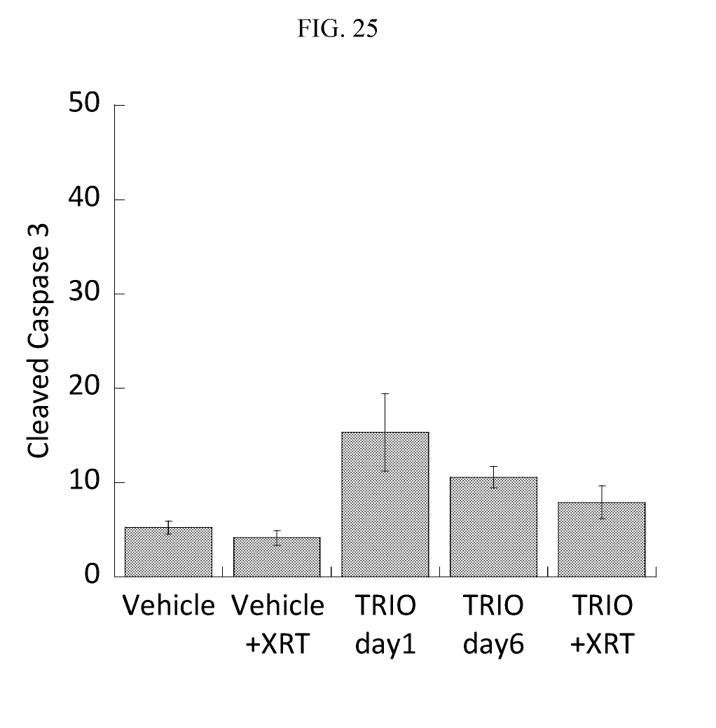

Levels of cleaved caspase 3 as a measure of apoptosis are shown in FIGS. 23-25. The greatest cleaved caspase 3 levels were observed in the P/R treatment group (FIG. 23). Although Triolimus (FIG. 24) and TRIO+XRT treatment (FIG. 25) slightly augmented the level of cleaved caspase 3, these effects were much less than the effect of P/R treatment.

Immunohistochemical staining for AKT, phosphorylated AKT (pAKT) and HSP70 was performed with the appropriate controls. The results were marginally different between the treatment groups, suggesting that the individual differences in protein expression might be below the resolution of qualitative immunohistochemistry. Moderate, diffuse expression of AKT was seen in the vehicle groups as well as in PTX, Low TRIO, Int. TRIO, High TRIO, TRIO+XRT, and TRIO day 6 tumors (Table 3). Interestingly in P/17, P/R, and 17/R, AKT expression was mostly weak. Expression of pAKT was essentially negative in 17/R groups and variably focal in other treatment groups without significant individual differences. HSP70 showed strong diffuse immunostaining in all groups (data not shown).

TABLE 3
Expression of AKT and pAKT in treatment
group (positive/total cases)

| Study group | AKT | pAKT |
|---|---|---|
| Vehicle | 8/8, diffuse mod | 7/8, weak(5), mod(2) |
| PTX | 4/4 diffuse, mod | 0/4 |
| P/17 | 4/4 diffuse, weak | 4/4 focal weak(3), mod(1) |
| P/R | 4/4 diffuse weak(3), mod(1) | 4/4 focal weak(3), mod(1) |
| 17/R | 4/4 diffuse weak(3), mod(1) | 0/4 |
| Low TRIO | 4/4 diffuse, mod | 3/4 focal mod(1), weak(2) |
| Int. TRIO | 4/4 diffuse, mod | 4/4 focal mod(3), weak(1) |
| High TRIO | 4/4 diffuse, mod | 2/4 focal mod(1), weak(1) |
| TRIO day 6 | 4/4 diffuse, mod | 3/4 focal mod |
| Vehicle + XRT | 4/4 diffuse mod(2), | 2/4 focal mod |
| | strong(2) | |
| TRIO + XRT | 4/4 diffuse, mod | 3/4 focal mod(2), weak(1) |

Mod = moderate.

### Example 10

### Hematologic, Metabolic and Histologic Analysis of Drug Toxicity

Following General Procedure 5, hematologic, metabolic, and histologic analysis of drug toxicity was performed. Histologic sections of mouse heart, lungs, liver, spleen, kidneys and brain were examined under a light microscope to morphologically assess organ toxicity. Most tissues revealed no significant pathologic abnormalities. There was very focal mild lymphocytic interstitial inflammation in the lungs of six animals (two in the vehicle group, one in the Int. TRIO group, two in the Low TRIO group, and one in the P/17 group). Twenty-three animals showed minimal non-specific periportal chronic inflammation, including six animals in the vehicle group and two in the vehicle+XRT group. These changes are of uncertain significance but given that they are minimal and present in a large number of control animals, they are unlikely to be treatment related.

Comprehensive metabolic and hematologic analyses following dosing with vehicle, PTX, dual drug combinations, TRIO, and TRIO+XRT are summarized in Table 4. PTX treatment significantly reduced white blood cell counts. Statistically significant reduction in hemoglobin was observed in Int. TRIO group. Statistically significant reductions in white blood cell count were observed in Low TRIO and P/R treatment groups. Moreover, statistically significant reductions in red blood cell count were also observed in P/R treatment group. Statistically significant reductions in white blood cell count were seen in TRIO+XRT and vehicle+XRT groups. However, this effect was no longer evident in the TRIO group on day six. This suggests that the marked reduction of white blood cell count is likely caused by radiation. The effects on red blood cells also appear to be mainly caused by radiation since statistically significant reductions in red blood cell counts, hemoglobin, and hematocrit were observed in the vehicle+XRT treatment group. Slight reductions in red blood cell count, hemoglobin and hematocrit were also observed in the TRIO+XRT treatment group indicating that the adverse effect of radiation was not increased by combining it with Triolimus. Kidney function was assessed by measurements of plasma electrolytes, creatinine, and blood urea nitrogen. Almost all treatment groups except P/17 and 17/R had statistically significant increases in urea nitrogen. Significant differences in creatinine levels were not observed in any treatment group. Also, some of electrolyte levels were statistically increased after drug or XRT treatment, specifically sodium levels in both P/17 and 17/R treated groups and chloride levels in the P/R treated group. These results suggest that drug and/or XRT treatment may slightly perturb renal function or may result in slight dehydration.

TABLE 4
Hematological analysis of drug and radiation treated mice

| | | | | | | P/17 |
|---|---|---|---|---|---|---|
| RBC [10⁶/μL] | 8.8 ± 0.2 | 8.3 ± 0.3 | 8.5 ± 0.1 | 8.7 ± 0.2 | 8.4 ± 0.1 | 8.0 ± 0.3 |
| HGB [g/dL] | 14.7 ± 0.4 | 13.9 ± 0.3*** | 14.2 ± 0.2 | 15.3 ± 0.5 | 14.9 ± 0.2 | 14 ± 1 |
| HCT [%] | 43 ± 1 | 42 ± 1 | 42 ± 1 | 45 ± 1 | 43 ± 1 | 42 ± 2 |
| PLATELET [10³/μL] | 960 ± 50 | 1049 ± 45 | 1146 ± 92 | 643 ± 194 | 902 ± 122 | 702 ± 158 |
| WBC [10³/μL] | 5.3 ± 0.5 | 9.4 ± 3.3 | 8.0 ± 0.9*** | 4.5 ± 0.6 | 2.7 ± 0.2*** | 3.5 ± 0.9 |
| Sodium [mmol/L] | 145 ± 1 | 150 ± 0 | 146 ± 1 | 146 ± 1 | 143 ± 1 | 142 ± 0.3*** |
| Potassium [mmol/L] | 3.8 ± 0.3 | 3.6 ± 0.3 | 3.2 ± 0.3 | 3.6 ± 0.1 | 3.6 ± 0.1 | 3.9 ± 0.3 |
| Chloride [mmol/L] | 112 ± 1 | 112 ± 0 | 113 ± 1 | 114 ± 1 | 116 ± 1 | 114 ± 1 |
| Total CO2 [mmol/L] | 17 ± 1 | 18 ± 1*** | 16 ± 1 | 15 ± 1 | 12.3 ± 0.3*** | 13 ± 1 |
| Anion Gap | 20 ± 1 | 19 ± 1 | 20 ± 2 | 21 ± 2 | 18.7 ± 0.3 | 18 ± 2 |
| Calcium [mg/dL] | 9.5 ± 0.1** | 9.0 ± 0.1 | 9.0 ± 0.1 | 8.9 ± 0.0 | 9.4 ± 0.0* | 9.4 ± 0.1*** |
| Phosphorus [mg/dL] | 6.4 ± 0.8 | 6.0 ± 0.4 | 5.2 ± 1.1 | 9.2 ± 1.7 | 8.4 ± 0.4 | 8.1 ± 0.8 |
| Magnesium [mg/dL] | 2.1 ± 0.1 | 2.5 ± 0.1 | 2.3 ± 0.1 | 2.2 ± 0.1 | 2.3 ± 0.1 | 2.3 ± 0.1 |
| Glucose [mg/dL] | 200 ± 16 | 183 ± 3 | 197 ± 10 | 149 ± 33 | 173 ± 12 | 199 ± 14 |
| Urea Nitrogen [mg/dL] | 22 ± 1* | 25 ± 1* | 22 ± 1* | 15.3 ± 0.3 | 23 ± 2*** | 17 ± 2 |
| Creatinine [mg/dL] | 0.3 ± 0.0 | 0.2 ± 0.0 | 0.2 ± 0.0 | 0.2 ± 0.0 | 0.2 ± 0.0 | 0.2 ± 0.0 |
| Total Protein [g/dL] | 4.5 ± 0.1*** | 4.3 ± 0.1 | 4.5 ± 0.1*** | 4.2 ± 0.1 | 4.4 ± 0.1 | 4.2 ± 0.2 |
| Albumin [g/dL] | 2.1 ± 0.1 | 2.0 ± 0.0 | 2.1 ± 0 | 2.0 ± 0.0 | 2.1 ± 0.1 | 2.1 ± 0.1 |
| Globulin [g/dL] | 2.4 ± 0.1 | 2.3 ± 0.1 | 2.4 ± 0.1 | 2.2 ± 0.1 | 2.3 ± 0.1 | 2.1 ± 0.1 |
| AST [U/L] | 137 ± 10*** | 170 ± 31*** | 86 ± 5 | 88 ± 12 | 97 ± 7 | 113 ± 5 |
| ALT [U/L] | 51 ± 3 | 50 ± 5 | 43 ± 2 | 46 ± 1 | 50 ± 2 | 55 ± 3 |
| Alkaline Phosphatase [U/L] | 74 ± 8 | 62 ± 7 | 75 ± 11 | 63 ± 4 | 68 ± 9 | 80 ± 10 |
| Total Bilirubin [mg/dL] | N.D. | N.D. | N.D. | N.D. | N.D. | N.D. |
| Cholesterol [mg/dL] | 86 ± 5 | 87 ± 5 | 92 ± 4*** | 78 ± 4 | 111 ± 4*** | 91 ± 10 |
| Triglycerides [mg/dL] | 98 ± 18 | 117 ± 9 | 105 ± 18 | 93 ± 3 | 181 ± 32 | 82 ± 5 |

| | P/R | 17/R | TRIO + XRT | Vehicle + XRT | TRIO Day 6 |
|---|---|---|---|---|---|
| RBC [10⁶/μL] | 8.0 ± 0.1*** | 8.4 ± 0.3 | 8.5 ± 0.4 | 7.9 ± 0.2*** | 8.3 ± 0.4 |
| HGB [g/dL] | 14.1 ± 0.1 | 14.5 ± 0.5 | 11 ± 3 | 13.3 ± 0.3*** | 13.9 ± 0.4 |
| HCT [%] | 41.8 ± 0.3 | 44 ± 1 | 42 ± 2 | 39 ± 1** | 42 ± 1 |
| PLATELET [10³/μL] | 755 ± 83 | 1153 ± 109 | 765 ± 61 | 1030 ± 108 | 1428 ± 3 |
| WBC [10³/μL] | 2.1 ± 0.2** | 6.7 ± 0.9 | 0.8 ± 0.0** | 0.8 ± 0.0*** | 5.8 ± 1.6 |
| Sodium [mmol/L] | 144 ± 1 | 140 ± 1** | 146 ± 1 | 146 ± 1 | 148 ± 1 |
| Potassium [mmol/L] | 3.6 ± 0.1 | 3.7 ± 0.3 | 4.0 ± 0.2 | 4.0 ± 0.3 | 3.7 ± 0.1 |
| Chloride [mmol/L] | 118 ± 1** | 113 ± 1 | 113 ± 1 | 114 ± 0 | 115 ± 0.4 |
| Total CO2 [mmol/L] | 11 ± 1*** | 14.0 ± 0.4 | 17 ± 3 | 12.5 ± 0.5*** | 10 ± 1* |
| Anion Gap | 19.0 ± 0.4 | 17 ± 1 | 20 ± 2 | 24 ± 1 | 26 ± 1*** |
| Calcium [mg/dL] | 8.9 ± 0.1 | 9.4 ± 0.2 | 9.2 ± 0.1 | 8.4 ± 0.2*** | 8.8 ± 0.1 |
| Phosphorus [mg/dL] | 8.0 ± 0.7 | 6.7 ± 0.3 | 6.7 ± 0.4 | 6.4 ± 0.3 | 7.1 ± 0.6 |
| Magnesium [mg/dL] | 2.3 ± 0.0 | 1.9 ± 0.1 | 2.2 ± 0.1 | 2.3 ± 0.1 | 2.3 ± 0.1 |
| Glucose [mg/dL] | 175 ± 11 | 194 ± 12 | 170 ± 18 | 165 ± 17 | 187 ± 20 |
| Urea Nitrogen [mg/dL] | 24 ± 1* | 17 ± 1 | 17.7 ± 0.3** | 25 ± 2*** | 20.3 ± 0.6* |
| Creatinine [mg/dL] | 0.2 ± 0.0 | 0.2 ± 0.0 | 0.3 ± 0.0 | 0.2 ± 0.0 | 0.2 ± 0.0 |
| Total Protein [g/dL] | 4.1 ± 0.1 | 4.3 ± 0.1 | 4.8 ± 0.2*** | 4.2 ± 0.1 | 4.4 ± 0.1 |
| Albumin [g/dL] | 2.0 ± 0.1 | 2.1 ± 0.0 | 2.1 ± 0.1 | 2.0 ± 0.1 | 2.0 ± 0.1 |
| Globulin [g/dL] | 2.1 ± 0.1 | 2.2 ± 0.0 | 2.8 ± 0.1*** | 2.2 ± 0.1 | 2.4 ± 0.1 |
| AST [U/L] | 97 ± 18 | 90 ± 14 | 125 ± 26 | 86 ± 20 | 58 ± 6 |
| ALT [U/L] | 52 ± 3 | 45 ± 1 | 42 ± 3 | 45 ± 4 | 38 ± 1* |
| Alkaline Phosphatase [U/L] | 71 ± 7 | 66 ± 4 | 60 ± 2 | 84 ± 8 | 67 ± 3 |
| Total Bilirubin [mg/dL] | N.D. | N.D. | N.D. | N.D. | N.D. |
| Cholesterol [mg/dL] | 88 ± 4 | 101 ± 1* | 126 ± 9** | 73 ± 1 | 96 ± 6 |
| Triglycerides [mg/dL] | 121 ± 4* | 128 ± 16 | 202 ± 46 | 99 ± 14 | 189 ± 44 |

*Significantly different from vehicle group (P < 0.005)
**Significantly different from vehicle group (P < 0.01)
***Significantly different from vehicle group (P < 0.05)
RBC: Red blood cell count,
HGB: Hemoglobin,
HCT: Hematocrit,
WBC: White blood cell count

Liver function was assessed by measuring glucose, total protein, albumin, globulin, AST, ALT, alkaline phosphatase, and total bilirubin levels. No significant differences were observed in glucose, albumin, ALT, or alkaline phosphatase levels in any group. Total bilirubin was undetectable in each sample. However, total protein level in High TRIO, Low TRIO, and TRIO+XRT treatment groups, globulin level in the TRIO+XRT treatment group, and AST levels in High TRIO and Int. TRIO treatment groups were significantly increased. Thus, it appears that Triolimus with or without XRT treatment may impact liver function.

Significant difference of total CO₂ level in Int. TRIO, PTX, P/R, vehicle+XRT, and TRIO day 6 treatment group, and anion gap in TRIO day 6 group were also noted, potentially suggesting altered pulmonary function.

In general the alterations to body chemistry for Triolimus were comparable to PTX alone and dual drug treated groups. Testing the TRIO group on day six revealed that changes in body chemistries and complete blood counts were short-lived; all values except those for CO₂, anion gap and urea nitrogen were not significantly different from those in the vehicle treated group.

1. A method of treatment comprising administering to lung, brain or esophageal cancer cells an effective amount of radiation in conjunction with a micelle composition comprising an effective amount of paclitaxel, and one or both of 17-AAG, and rapamycin, wherein the micelle comprises poly(ethylene glycol)-block-poly(lactic acid) (PEG-b-PLA).

2. The method of claim 1, wherein the radiation and the micelle composition are administered sequentially, simultaneously, or separately to the cancer cells.

3. The method of claim 1, wherein the radiation is administered over 1-5 days sequentially, simultaneously, or separately with the micelle.

4. The method of claim 1, wherein radiation is administered at a dosage between 1 Gy/day to 8 Gy/day.

5. The method of claim 1, wherein drug loading of paclitaxel, 17-AAG, and rapamycin in the micelles is about 5 wt. % to about 40 wt. %.

6. The method of claim 1, wherein the PEG-b-PLA comprises a poly(ethylene glycol)-block having a molecular weight of about 1,500 to about 30,000 g/mol and a poly(lactic acid) block having a molecular weight of about 1,500 to about 30,000 g/mol.

7. The method of claim 1, wherein the micelle composition comprises a ratio by mass of paclitaxel, 17-AAG, and rapamycin of about 2:2:1 to about 5:1:1.

8. The method of claim 1, wherein the micelle composition comprises a ratio by mass of paclitaxel, 17-AAG, and rapamycin of about 2:2:1.

9. The method of claim 1, wherein radiation and the micelle composition are administered to lung, brain or esophageal cancer cells in vitro.

10. The method of claim 1, wherein the lung, brain or esophageal cancer cells are in a subject.

11. The method of claim 11, wherein the subject is human.

12. The method of claim 11, wherein the micelle composition comprises an aqueous carrier and is administered to the subject intravenously or intraperitoneally.

13. The method of claim 13, wherein the aqueous carrier comprises saline or an aqueous carbohydrate solution.

14. The method of claim 1, wherein the lung cancer cells are non-small cell lung cancer cells.

15. The method of claim 14, wherein the non-small cell lung cancer cells are adenocarcinoma, squamous, or large cell lung cancer cells.

16. The method of claim 1, wherein the lung cancer cells are small cell lung cancer cells.

17. The method of claim 1, wherein the micelle composition comprises an effective amount of paclitaxel, 17-AAG, and rapamycin.

18. The method of claim 1, wherein the micelle composition comprises an effective amount of paclitaxel and rapamycin.

19. The method of claim 1, wherein the micelle composition comprises an effective amount of paclitaxel and 17-AAG.

20. A method of treatment comprising administering to a subject having lung, brain or esophogeal cancer an effective amount of radiation and an effective amount of a micelle composition comprising paclitaxel, and one or both of 17-AAG, and rapamycin, wherein the micelle composition comprises poly(ethylene glycol)-block-poly(lactic acid) polymers.
